(19) United States
(12) Patent Application Publication
(10) Pub. No.: US 2011/0212955 A1
(43) Pub. Date: Sep. 1, 2011

(54) ROSAMINE DERIVATIVES AS AGENTS FOR THE TREATMENT OF CANCER

(75) Inventors: Hong Boon Lee, Subang Jaya (MY); Kevin Burgess, College Station, TX (US); Siang Hui Lim, Subang Jaya (MY); Liangxing Wu, College Station, TX (US)

(73) Assignee: CANCER RESEARCH INITIATIVES FOUNDATION, Subang Jaya, Selangor (MY)

(21) Appl. No.: 13/119,904

(22) PCT Filed: Jul. 2, 2009
(86) PCT No.: PCT/MY2009/000090

§ 371 Date: May 3, 2011

(30) Foreign Application Priority Data
Sep. 19, 2008 (MY) PI 20083675

(51) Int. Cl. A61K 31/5377; C07D 405/14; A61K 31/4025; A61K 31/4545; C07D 413/14; A61K 31/496; C07D 405/04; C07D 413/04; A61P 35/00; A61P 35/02

(52) U.S. Cl. 514/232.5; 548/523; 514/422; 546/187; 514/316; 544/129; 544/79; 514/232.8; 544/357; 514/252.11; 544/130; 548/525; 546/196; 544/150; 544/375

(57) ABSTRACT

The present invention relates to a new class of rosamine derivatives, in one embodiment, the compounds have the structure (I) or any pharmaceutically acceptable salt or solvate thereof, wherein: R¹ represents aryl, Het¹ or C₁₋₆ alkyl, which latter group is optionally substituted by aryl or Het²; R^{2a} and R^{2b} together form C_{3.8} n-alkylene, which alkylene group is optionally substituted by one or more substituents selected from halo, C₁₋₄ alkyl, C(O)OH and C(O)O—C₁₋₄, alkyl and which alkylene group is optionally interrupted by X¹; R^{3a} and R^{3b} together form C_{3-6 /7-alkylene, which alkylene group is} optionally substituted by one or more substituents selected from halo. C₁₋₄ alkyl, C(O)OH and C(O)O—C₁₋₄ alkyl, and which alkylene group is optionally interrupted by X²; X¹ and X² independently represent O, S, or NR⁴; R⁴ represents, independently at each occurrence, H, C(O)OR⁵, C(O)R^{6a}, C(O)N(R^{6b})R^{6c} or C₁₋₆, alkyl, which latter group is optionally substituted by one or more substituents selected from halo, aryl and Het³ or is substituted by a single C(O)OR^{1a} group; R^{4a} represents H or C₁₋₄ alkyl; R⁵ represents aryl, Het⁴ or C₁₋₆ alkyl optionally substituted by one or more substituents selected from halo, aryl and Het⁵; R^{5e} to R^{6d} independently represent H or R⁵; each aryl independently represents a C₆₋₁₀carbocylic aromatic group, which group may comprise either one or two rings and may be substituted by one or more substituents selected from halo, CN, C₁₋₆ alkyl (which latter group is optionally substituted by one or more substituents selected from halo, OR⁷, phenyl, napthyl and Het⁶) and OR⁸; R⁷ and R⁸ independently represent H, C₁₋₄ alkyl (optionally substituted by one or more halo groups or by a single phenyl or C(O)OR^{8a} substituent), Het⁷, phenyl or naphthyl; R^{8a} represents H or C₁₋₄ alkyl; Het¹ to Het⁷ independently represent 5- to 10-membered aromatic, fully saturated or partially unsaturated heterocyclic groups containing one or more heteroatoms selected from oxygen, nitrogen and/or sulphur, which heterocyclic groups may comprise one or two rings and may be substituted by one or more substituents selected from Halo, CN, C₁₋₆ alkyl (which latter group is optionally substituted by one or more substituents selected from halo, OR⁹ and phenyl) and OR¹⁰; R⁹ and R¹⁰ independently represent H, C₁₋₄ alkyl or phenyl; unless otherwise specified, alkyl groups are optionally substituted by one or more halo atoms; and A′ represents a pharmaceutically acceptable anion. Also disclosed are methods for making and using compounds as well as pharmaceutical compositions.

### FIELD OF THE INVENTION

The present invention relates to a new class of rosamine derivatives and their use for treating cancer.

### BACKGROUND OF THE INVENTION

Conventional chemotherapy in cancer treatment depends largely on drugs that act by interrupting DNA replication, that is by inhibiting the synthesis or function of new nucleic materials, or by causing irreparable damage to vital nucleic acids through intercalation, alkylation or enzymatic inhibition mechanisms. These drugs typically target rapidly dividing cells but lack selectivity for neoplastic cells which leads to limited success in cancer treatment. Therefore, it is important to investigate other cellular targets that are distinctly for normal cells and cancer cells to provide a basis for selective tumour cell killing.

Mitochondria are the main energy generators that maintain cell life and essential cell functions. There is evidence to show that they are also involved in diverse cellular events by being an integral part of multiple signaling cascades in regulation of metabolism, cell-cycle control, development, antiviral responses and cell death (Heidi et al. 2006). As a powerhouse, mitochondria generate energy through oxidative phosphorylation where oxidation of respiratory substrates is coupled to the synthesis of ATP under aerobic conditions. This process involves a sequence of electron transfers from respiratory substrates to oxygen, concurrent with proton translocation from the mitochondrial inner compartment to the intermembrane space through a series of respiratory chain complexes located on the inner membrane. The electrochemical proton gradient thus formed, also designated as the proton motive force, is the driving force for ATP synthesis through the back flow of protons through the ATP synthase complex. Importantly, this mitochondrial transmembrane proton-motive force which results in a negative potential inside the mitochondrial matrix selectively accumulates lipophilic cations which are membrane-permeable compounds with cationic characteristics (Szewczyk and Wojtczak, 2002). High concentrations of lipophilic cations in mitochondria often results in cell death by decreasing cellular ATP production, rendering mitochondria a unique target for cellular toxicity.

Studies have shown that the mitochondrial membrane potential of carcinoma cells are higher than in normal epithelial cells and that the accumulation and retention of lipophilic cations correlated with the mitochondrial membrane potential (Johnson et al. 1981; Nadakavukaren et al. 1985; Lampidis et al. 1985; Modica-Napolitano and Aprille 1987). This increase in mitochondrial membrane potential in carcinoma cells which leads to selective accumulation of toxic lipophilic cations provides a rationale for selective chemotherapy of cancer cells. Rhodamine 123 (Rh123) was the first example of lipophilic cation to exhibit selective anti-tumour activity. In in vitro experiments, this compound markedly induced cell death in 9/9 of carcinoma cell types while 6/6 of non-tumorigenic epithelial cell types remained unaffected when tested at similar concentrations (Lampidis et al. 1983). Other examples include the dequalinium chloride (Weiss et al. 1987), the thiopyrylium AA1 (Sun et al. 1994) and the thiatelluracarbocyanine iodide (Sun et al 1996) which demonstrated 10- to 100-fold greater inhibition of the clonal growth of carcinoma versus control epithelial cells in culture and anti-carcinoma activity in a number of whole animal tumor models. In more recent studies, a pyridinium cation codenamed F16 was identified through a high-throughput chemical library screen as a small molecule that selectively inhibited proliferation of a variety of transformed mouse mammary epithelial cells which had correlated increase in mitochondrial transmembrane potential (Fantin et al. 2002). An intraperitoneal injection of F16 was observed to retard the growth of A6-derived subcutaneous tumors in nude mice. In a separate example, a rhodacyanine dye known as MKT-077 was shown to significantly inhibit growth of cancer cells in vitro and in vivo, leading to its approval as a mitochondria-targeting lipophilic cation for treatment of carcinoma in clinical trials. (Kawakami et al. 1998; Proper et al. 1999; Britten 2000)

Although the clinical trials were discontinued in phase II due to a lack of efficacy at the particular approved dosage and drug regimen, the study established that MKT-077 was preferentially accumulated in tumor cell mitochondria.

In spite of the potential of lipophilic cations in selectively targeting cancer cells in therapeutic settings, there has not been further report from this class of compounds as new candidates for targeted cancer therapy.

### SUMMARY OF THE INVENTION

According to a first aspect of the invention, there is provided a compound of formula I:

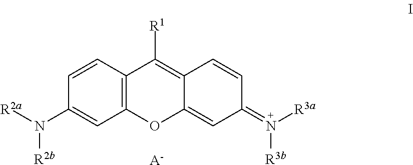

or any pharmaceutically acceptable salt or solvate thereof, wherein:
R¹ represents aryl, Het¹ or C₁₋₆ alkyl, which latter group is optionally substituted by aryl or Het²;
R^{2a} and R^{2b} together form C₃₋₆ n-alkylene, which alkylene group is optionally substituted by one or more substituents selected from halo, C₁₋₄ alkyl, C(O)OH and C(O)O—C₁₋₄ alkyl, and which alkylene group is optionally interrupted by X¹;
R^{3a} and R^{3b} together form C₃₋₆ n-alkylene, which alkylene group is optionally substituted by one or more substituents selected from halo, C₁₋₄ alkyl, C(O)OH and C(O)O—C₁₋₄ alkyl, and which alkylene group is optionally interrupted by X²;
X¹ and X² independently represent O, S, or NR⁴;
R⁴ represents, independently at each occurrence, H, C(O)OR⁵, C(O)R^{6a}, C(O)N(R^{6b})R^{6c} or C₁₋₆ alkyl, which latter group is optionally substituted by one or more substituents selected from halo, aryl and Het³ or is substituted by a single C(O)OR^{4a} group;
R^{4a} represents H or C₁₋₄ alkyl;
R⁵ represents aryl, Het⁴ or C₁₋₆ alkyl optionally substituted by one or more substituents selected from halo, aryl and Het⁵;
R^{6a} to R^{6d} independently represent H or R⁵;
each aryl independently represents a C₆₋₁₀ carbocyclic aromatic group, which group may comprise either one or two rings and may be substituted by one or more substituents selected from halo, CN, C₁₋₆ alkyl (which latter group is optionally substituted by one or more substituents selected from halo, OR⁷, phenyl, naphthyl and Het⁶) and OR⁸;
R⁷ and R⁸ independently represent H, C₁₋₄ alkyl (optionally substituted by one or more halo groups or by a single phenyl or C(O)OR^{8a} substituent), Het⁷, phenyl or naphthyl;
R^{8a} represents H or C₁₋₄ alkyl;
Het¹ to Het⁷ independently represent 5- to 10-membered aromatic, fully saturated or partially unsaturated heterocyclic groups containing one or more heteroatoms selected from oxygen, nitrogen and/or sulfur, which heterocyclic groups may comprise one or two rings and may be substituted by one or more substituents selected from halo, CN, C₁₋₆ alkyl (which latter group is optionally substituted by one or more substituents selected from halo, OR⁹ and phenyl) and OR¹⁰;
R⁹ and R¹⁰ independently represent H, C₁₋₄ alkyl or phenyl;
unless otherwise specified, alkyl groups are optionally substituted by one or more halo atoms; and
A⁻ represents a pharmaceutically acceptable anion.

Pharmaceutically acceptable anions that may be mentioned include carboxylates (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate), halides (e.g. chloride, bromide or iodide), sulfonates (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate) or sulfate, pyrosulfate, bisulfate, sulfite, bisulfite, phosphate, monohydrogenphosphate, dihydrogenphosphate, metaphosphate, pyrophosphate or nitrate, and the like.

Alternatively, the pharmaceutically acceptable anion may be a negatively charged group (e.g. an −O⁻ group derived from an OH moiety) within the compound of formula I itself. In instances where the compound of formula I contains only one such negatively charged group (and only one positively charged group—i.e. the N-atom to which R^{3a} and R^{3b} are attached), the compound of formula I has no overall electrostatic charge. In this instance, the compound of formula I can be described as a zwitterion.

Alternatively, the pharmaceutically acceptable salts may be C₁₋₄ alkyl quaternary ammonium salts.

In one particular embodiment of the invention, the pharmaceutically acceptable anion is a halide (e.g. chloride) ion.

Pharmaceutically acceptable salts may be salts with acids or bases. Acid addition salts may be formed, for example, by protonation of a basic moiety within the compound of formula I (e.g. the tertiary N-atom to which R^{2a} and R^{2b} are attached, or a nitrogen-containing heterocyclic substituent). Acid addition salts that may be mentioned include salts with the acids containing the pharmaceutically acceptable anions described above.

Pharmaceutically acceptable solvates that may be mentioned include hydrates.

The term “halo”, when used herein, includes fluoro, chloro, bromo and iodo.

Heterocyclic (Het¹ to Het⁷) groups may be fully saturated, partly unsaturated, wholly aromatic or partly aromatic in character. Values of heterocyclic (Het¹ to Het⁷) groups that may be mentioned include 1-azabicyclo-[2.2.2]octanyl, benzimidazolyl, benzo[c]isoxazolidinyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo-[c]furanyl, 1,3-dihydro-2,1-benzisoxazolyl, 2,3-dihydropyrrolo[2,3-b]pyridinyl, dioxanyl, furanyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, oxadiazolyl, 1,2- or 1,3-oxazinanyl, oxazolyl, phthalazinyl, piperazinyl, piperidinyl, purinyl, pyranyl, pyrazinyl, pyrazolyl, pyridazinyl, pyridinyl, pyrimidinyl, pyrrolidinonyl, pyrrolidinyl, pyrrolinyl, pyrrolo[2,3-b]pyridinyl, pyrrolo[5,1-b]pyridinyl, pyrrolo[2,3-c]pyridinyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydro-benzo[e]pyrimidine, tetrahydrofuranyl, tetrahydropyranyl, 3,4,5,6-tetrahydropyridinyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridinyl, thiochromanyl, triazolyl, 1,3,4-triazolo[2,3-b]pyrimidinyl and the like.

Values of Het¹ that may be mentioned include thienyl (e.g. thien-2-yl).

Compounds of formula I may exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Embodiments of the invention that may be mentioned include those in which:

R¹ represents methyl (which latter group is optionally substituted by phenyl, which phenyl group is optionally substituted by one or two substituents selected from halo, C₁₋₄ alkyl and OR⁸), aryl or Het¹;
R^{2a} and R^{2b} together represent uninterrupted C₄₋₅ n-alkylene or C₃₋₄ n-alkylene interrupted by X¹ (e.g. R^{2a} and R^{3a} together represent —(CH₂)₄₋₅— or —(CH₂)₁₋₂—X¹—(CH₂)₂—);
R^{3a} and R^{3b} together represent uninterrupted C₄₋₅ n-alkylene or C₃₋₄ n-alkylene interrupted by X² (e.g. R^{3b} and R^{3a} together represent —(CH₂)₄₋₅— or —(CH₂)₁₋₂—X²—(CH₂)₂—);
X¹ and X² independently represent O or NR⁴;
R⁴ represents, independently at each occurrence, H, C(O)OR⁵ or methyl, which latter group is optionally substituted by one or more halo substituents or is substituted by a single C(O)OR^{4a} group;
R⁵ represents C₁₋₄ alkyl (e.g. t-butyl);
each aryl independently represents phenyl or naphthyl, which group may be substituted by one or more substituents selected from halo, C₁₋₄ alkyl and OR⁸;
R⁸ represents H or methyl (which latter group is optionally substituted by a single C(O)OR^{8a} substituent);
R^{8a} represents H or C₁₋₄ alkyl;
Het¹ represents a 5- or 6-membered aromatic, heterocyclic groups containing one to three heteroatoms selected from oxygen, nitrogen and/or sulfur, which heterocyclic group may be substituted by one or more substituents selected from halo and C₁₋₄ alkyl;
A⁻ represents a halide (e.g. chloride) ion.

Further embodiments of the invention that may be mentioned include those in which:

R¹ represents methyl, benzyl, phenyl (which latter group is optionally substituted by one or two substituents selected from C₁₋₂ alkyl, halo (e.g. iodo) and C₁₋₂ alkoxy) or thienyl;
R^{2a} and R^{2b} together represent —(CH₂)₂—X¹—(CH₂)₂— or, particularly, —(CH₂)₄— or —(CH₂)₅—;
R^{3a} and R^{3b} together represent —(CH₂)₄—, —(CH₂)₅— or —(CH₂)₂—X²—(CH₂)₂—;
X¹ and X² independently represent O or NR⁴;
R⁴ represents, independently at each occurrence, H or, particularly, C(O)OR⁵.

Still further embodiments of the invention that may be mentioned include those in which:

R¹ represents methyl, 2,6-dihydroxyphenyl, 2,6-bis(carboxymethoxy)phenyl or, particularly, benzyl, thienyl (e.g. thien-2-yl), phenyl, 2-methylphenyl, 2-methoxyphenyl, 4-iodophenyl or 4-methoxyphenyl;
R^{2a} and R^{2b} together represent —(CH₂)₂—O—(CH₂)₂—, —(CH₂)₂—NH—(CH₂)₂— or, particularly, —(CH₂)₄—, —(CH₂)₅— or —(CH₂)₂—N(C(O)O-t-butyl)-(CH₂)₂—;
R^{2a} and R^{2b} together represent —(CH₂)₂—NH—(CH₂)₂— or, particularly, —(CH₂)₄—, —(CH₂)₅—, —(CH₂)₂—O—(CH₂)₂— or —(CH₂)₂—N(C(O)O-t-butyl)-(CH₂)₂—.

Compounds of formula I may possess pharmacological activity and/or useful spectroscopic (e.g. fluorescence) properties. Thus, second and third aspects of the invention relate to:

(a) compounds of formula I for use in medicine; and
(b) compounds of formula I for use as a dye or chromophore (e.g. fluorophore).

In relation to (b) above, another aspect of the invention relates to a method of dyeing a substrate (e.g. a synthetic or natural fabric), the method comprising contacting the substrate with a compound of formula I, as hereinbefore defined.

When employed as a chromophore, the compounds of formula I may either be used directly or in chemically modified form. Chemical modifications to the compounds of formula I that may be mentioned include chemical conjugation to a substrate moiety (e.g. a moiety selected from the group consisting of amino acids, amino acid oligomers and polymers, proteins, nucleosides, nucleotides, polynucleotides, carbohydrates, ligands, particles, solid surfaces, organic and inorganic polymers and combinations or assemblages thereof, such as chromosomes, nuclei, living cells and the like).

Chemical conjugation of compounds of formula I may be achieved, for example, by attaching a linker group to appropriate functional groups on the compound of formula I and the substrate moiety. Appropriate functional groups on the compounds of formula I include, for example, OH, NH and C(O)OH groups.

Thus, a further aspect of the invention relates to compounds of formula Ia,

(I)-G-L-(Substrate) Ia

wherein:
(I)-G- represents a compound of formula I, as hereinbefore defined, possessing at least one functional group G, wherein G represents OH, NH or C(O)OH;
L represents a linker group consisting of from 1 to 30 atoms selected from C, N, O and S, said linker group containing at least one C-atom and the appropriate number of H-atoms needed to satisfy valency requirements; and
(Substrate) represents a substrate moiety (e.g. a moiety selected from the group consisting of amino acids, amino acid oligomers and polymers, proteins, nucleosides, nucleotides, polynucleotides, carbohydrates, ligands, particles, solid surfaces, organic and inorganic polymers and combinations or assemblages thereof, such as chromosomes, nuclei, living cells and the like).

The substrate moiety may be connected to the linker group by any chemical linkage. However, when the substrate is a protein, the linker is, in certain embodiments of the invention, attached to the protein via a free OH, SH, NH or, particularly, NH₂ group (e.g. from a serine, tyrosine, cysteine, tryptophan, lysine or N-terminal amino acid in the peptide).

Linker groups that may be mentioned include C₁₋₁₀ alkylenecarbonyl groups (e.g. methylenecarbonyl). Such linker groups may be introduced by using, for example a halo-substituted alkyl carboxylic acid starting material, or an activated (e.g. carbonyl halide or N-hydroxysuccinimide ester) or protected (e.g. t-butyl or benzyl ester) derivative thereof. For instance, t-butylbromoacetate may be used as a starting material to introduce a methylenecarbonyl linker.

Methods of coupling linkers to substrates and to functional groups such as OH, NH and C(O)OH are well known to those skilled in the art. For example, peptide coupling techniques can be used to connect NH and C(O)OH groups (the NH group coming from either the linker, the compound of formula I or the substrate), and such techniques include, for example, coupling in the presence of a coupling agent (such as: oxalyl chloride in N,N-dimethylformamide; 1-(3-dimethylaminopropyl)-3-ethylcarbodiimide hydrochloride; dicyclohexyl carbodiimide; diisopropylcarbodiimide; [N,N,N′,N′-tetramethyl-O-(benzotriazol-1-yl)uronium hexafluorophosphate]; O-(azabenzotriazol-1-yl)-N,N,N′,N′-tetramethyluronium hexafluorophosphate; (benzotriazol-1-yloxy)tri-pyrrolidinophosphonium hexafluorophosphate; [N,N,N′,N′-tetramethyl-O-(benzotriazol-1-yl)uronium tetrafluoroborate]; and the like) and a suitable solvent (e.g. dichloromethane, acetonitrile, ethyl acetate or N,N-dimethylformamide), and optionally in the presence of a suitable catalyst (e.g. 1-hydroxybenzotriazole or N-hydroxysuccinimide) and/or an appropriate base (e.g. pyridine, 4-(N,N-dimethylamino)pyridine, triethylamine, 2,4,6-collidine or diisopropylethylamine). Further, coupling of OH, SH, NH or NH₂ groups to a haloalkyl moiety can be achieved in the presence of a suitable base (for example: an alkali metal carbonate such as sodium, potassium or caesium carbonte; or an alkali metal alkoxide, such as sodium methoxide or ethoxide) and an appropriate solvent (such as N,N-dimethylformamide), and optionally in the presence of a suitable catalyst (such as tetrabutylammonium iodide).

Compounds of formula Ia may find utility as optical probes (e.g. fluorescence probes) in a variety of different settings.

Further, according to a fourth aspect of the invention, there is provided a pharmaceutical composition comprising a compound of formula I, or any pharmaceutically acceptable salt or solvate thereof, and a pharmaceutically acceptable, carrier, adjuvant or vehicle.

When used in medicine, the compounds of formula I may be used as diagnostic tools or, particularly, as cytotoxic agents. Thus, according to fifth, sixth and seventh aspects of the invention, there is provided:
(a) a method of treating cancer in a patient in need of such treatment, the method comprising administering to the patient a therapeutically effective amount of a compound of formula I, or any pharmaceutically acceptable salt or solvate thereof;
(b) a compound of formula I, or any pharmaceutically acceptable salt or solvate thereof, for use in the treatment of cancer; and
(c) the use of a compound of formula I, or any pharmaceutically acceptable salt or solvate thereof, for the preparation of a medicament for the treatment of cancer.

When used herein, the terms “treating” and “treatment” are intended to encompass:
(a) curative treatment;
(b) ameliorating at least one symptom of the condition or disease; and
(c) prophylactic treatment.

For example, in the case of cancer, “treating” and “treatment” include, for example, achieving an increase in survival time, elongation in time to tumour progression, reduction in tumour mass, reduction in tumour burden and/or a prolongation in time to tumour metastasis.

When used herein, the term “therapeutically effective amount” is intended to refer to the amount of a compound or composition administered to the patient which is most likely to result in the desired response to treatment. The amount is empirically determined by the patient's clinical parameters (including, for example, one or more parameters selected from the age, gender and histology of the patient, and the stage of disease and likelihood of tumour recurrence).

When used herein, the term “patient” includes references to mammals (i.e. humans and non-human mammals).

In the fifth to seventh aspects of the invention, the cancer is preferably selected from the group consisting of leukemia and solid tumour cancers. Particular solid tumour cancers that may be mentioned include non-small cell lung cancer, small cell lung cancer, breast cancer, nasopharyngeal cancer, oral cancer, cancer of the pancreas, ovarian cancer, colorectal cancer, prostate cancer, gastric cancer, liver cancer, bladder cancer, cancer of the kidney, cervical cancer and cancer of the oesophagus.

When employed to treat cancer (e.g. according to any of the fifth to seventh aspects of the invention), the compounds of formula I may be employed as a sole anti-cancer agent (i.e. as a monotherapy) or in conjunction with one or more other anti-cancer agents.

Thus, according to an eighth aspect of the invention, there is provided a combination product comprising a compound of formula I, or any pharmaceutically acceptable salt or solvate thereof, and a known anti-cancer agent.

Known anti-cancer agents include those listed under the relevant headings in “Martindale: The Complete Drug Reference”, 32^{nd} Edition, the Pharmaceutical Press, London (1999), the disclosures of which document are hereby incorporated by reference.

Known anti-cancer agents also include non-chemical agents such as ionising radiation (e.g. subatomic particle radiation such as α-particles, β-particles, neutrons, protons, mesons and heavy ions or electromagnetic radiation such as high-frequency X-rays or gamma rays). Other known anti-cancer agents that may be mentioned include:

(a) Alkylating agents including:
(i) nitrogen mustards such as mechlorethamine (HN₂), cyclophosphamide, ifosfamide, melphalan (L-sarcolysin) and chlorambucil;
(ii) ethylenimines and methylmelamines such as hexamethylmelamine, thiotepa;
(iii) alkyl sulfonates and thiosulfonates such as busulfan, methyl methanesulfonate (MMS) and methyl methanethiosulfonate;
(iv) nitrosoureas and nitrosoguanidines such as carmustine (BCNU), lomustine (CCNU), semustine (methyl-CCNU), streptozocin (streptozotocin) and N-methyl-N′-nitro-N-nitrosoguanidine (MNNG); and
(v) triazenes such as dacarbazine (DTIC; dimethyltriazenoimidazole-carboxamide).
   (b) Antimetabolites including:
(i) folic acid analogues such as methotrexate (amethopterin);
(ii) pyrimidine analogues such as fluorouracil (5-fluorouracil; 5-FU), floxuridine (fluorodeoxyuridine; FUdR) and cytarabine (cytosine arabinoside); and
(iii) purine analogues and related inhibitors such as mercaptopurine (6-mercaptopurine; 6-MP), thioguanine (6-thioguanine; TG) and pentostatin (2′-deoxycoformycin).
   (c) Natural Products including:
(i) vinca alkaloids such as vinblastine (VLB) and vincristine;
(ii) epipodophyllotoxins such as etoposide and teniposide;
(iii) antibiotics such as dactinomycin (actinomycin A, C, D or F), daunorubicin (daunomycin; rubidomycin), doxorubicin, bleomycin, plicamycin (mithramycin) and mitomycin (mitomycin A, B or C);
(iv) enzymes such as L-asparaginase; and
(v) biological response modifiers such as interferon alphenomes.
   (d) Miscellaneous agents including:
(i) platinum coordination complexes such as cisplatin (cis-DDP) and carboplatin;
(ii) anthracenedione such as mitoxantrone and anthracycline;
(iii) substituted urea such as hydroxyurea;
(iv) methyl hydrazine derivatives such as procarbazine (N-methylhydrazine, MIH);
(v) adrenocortical suppressants such as mitotane (o,p′-DDD) and aminoglutethimide;
(vi) taxol and analogues/derivatives;
(vii) hormone agonists/antagonists such as flutamide and tamoxifen;
(viii) photoactivatable compounds (e.g. psoralens);
(ix) DNA topoisomerase inhibitors (e.g. m-amsacrine and camptothecin);
(x) anti-angiogenesis agents (e.g. SU6668, SU5416, combretastatin A4, angiostatin and endostatin); and
(xi) immunotherapeutic agents (e.g. radiolabelled antibodies such as Bexxar™ and Theragyn™ (Pemtumomab™)).

The combination product may be either a kit-of-parts or a combined preparation. Thus, the eighth aspect of the invention encompasses:

(a) a composition comprising
(I) a compound of formula I, or any pharmaceutically acceptable salt or solvate thereof,
(II) a known anti-cancer agent and, optionally
(III) a pharmaceutically acceptable, carrier, adjuvant or vehicle; or
   (b) a kit-of-parts comprising
(I) a first part which contains a compound of formula I, or any pharmaceutically acceptable salt or solvate thereof and, optionally a pharmaceutically acceptable, carrier, adjuvant or vehicle, and
(II) a second part which contains a known anti-cancer agent and, optionally a pharmaceutically acceptable, carrier, adjuvant or vehicle.

According to further aspects of the invention, there is provided
(a) a method of treating cancer in a patient in need of such treatment, the method comprising administering to the patient a therapeutically effective amount of a combination product according to the eighth aspect of the invention;
(b) a combination product according to the eighth aspect of the invention, for use in the treatment of cancer; and
(c) the use of a combination product according to the eighth aspect of the invention for the preparation of a medicament for the treatment of cancer.

When the compound of formula I, or any pharmaceutically acceptable salt or solvate thereof, is administered to a patient in combination with a used herein, the term “in combination with a known anti-cancer agent, the other agent may be administered, before, during and/or following administration of the compound of formula I.

Whether or not the compounds of formula I are directly cytotoxic, their physicochemical properties can allow those compounds to selectively accumulate in the mitochondria of cancer cells. The selective targeting of cancer cells by the compounds of formula I can be used to deliver other cytotoxic agents to cancer cells (e.g. by formation of a compound of formula Ia in which (Substrate) represents a cytotoxic agent).

Compounds of formula I have the advantage that they may have activity in the killing (e.g. selective killing) of cancer cells. This activity may be improved in comparison to known, structurally related compounds. Compounds of formula I may also have the advantage of possessing useful or improved spectroscopic properties (e.g. high fluorescence intensity and/or quantum yields, pH-dependent fluorescence properties, etc.).

Additionally, compounds of formula I have the advantage that they may be more efficacious, be less toxic, be longer acting, have a broader range of activity, be more selective (e.g. by targeting tumour cells rather than normally functioning cells), be more potent, produce fewer side effects, be more easily absorbed, and/or have a better pharmacokinetic profile (e.g. higher oral bioavailability and/or lower clearance), be more readily and conveniently synthesised than, and/or have other useful pharmacological, physical, or chemical, properties over, compounds known in the prior art.

Further, compounds of formula Ia have the advantage that they may allow for improved detection (e.g. through high quantum yields, greater sensitivity, etc.) of various physical phenomena (e.g. biological reactions, solution pH, solvent polarity, etc.), or they may be more readily and conveniently synthesised than other compounds known in the prior art.

Compounds of formula I may be prepared in accordance with known techniques or by the methods described below.

According to a further aspect of the invention, there is provided a method of preparing compounds of formula II,

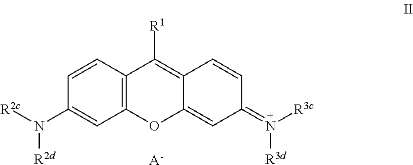

wherein R¹ is as hereinbefore defined
R^{2c}, R^{2d}, R^{3c} and R^{3d} independently represent C₁₋₆ alkyl (optionally substituted by one or more substituents selected from halo, OR^{a}, N(R^{b})R^{c}, aryl and Het¹,
or R^{2c} and R^{2d} together take the same definition as R^{2a} and R^{2b}, as hereinbefore defined and/or R^{3c} and R^{3d} together take the same definition as R^{3a} and R^{3b}, as hereinbefore defined,
R^{a} to R^{c} independently represent H, C₁₋₆ alkyl (optionally substituted by one or more halo groups or by one substituent selected from OH, aryl and Het²), aryl and Het³, aryl, Het¹ to Het³ and A⁻ are as hereinbefore defined,
which process comprises:
(a) reacting a compound of formula III

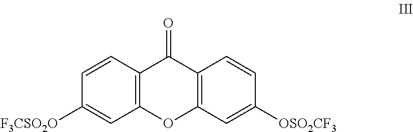

with at least one equivalent each of compounds of formulae IVa and IVb

R^{2c}(R^{2d})N—H IVa

R^{3c}(R^{3d})N—H IVa

wherein R^{2c}, R^{2d}, R^{3c} and R^{3d} are as defined above;
(b) reacting the resulting intermediate of formula V

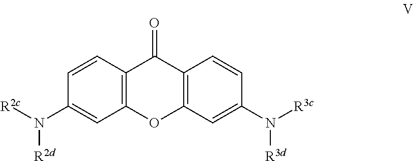

with a compound of formula VIa or VIb

R¹—Mg-Hal VIa

R¹—Li VIb

wherein Hal represents a halogen (e.g. Br) and R¹ is as hereinbefore defined; and then
(c) reacting the resulting intermediate of formula VII

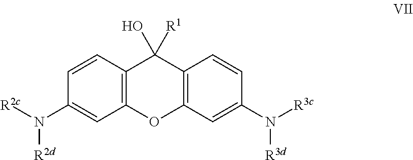

with acid H⁺A⁻, wherein A⁻ is as hereinbefore defined.

When R^{2c} and R^{2d} together take the same definition as R^{2a} and R^{2b}, as hereinbefore defined and R^{3c} and R^{3d} together take the same definition as R^{3a} and R^{3b}, as hereinbefore defined, then the process results in compounds of formula I, as hereinbefore defined.

In step (a) of the process, reaction of the compound of formula III with the compound of formula IVa may take place before, after or at the same time as reaction with the compound of formula IVb. That is, when the compounds of formulae IVa and IVb are the same, then the groups R^{2c}(R^{2d})N— and R^{3c}(R^{3d})N— may be introduced simultaneously.

Alternatively, when the compounds of formulae IVa and IVb are not the same, then the compound of formula III can be:
(i) reacted with at least one equivalent of a compound of formula IVa to provide the intermediate of formula IIIa

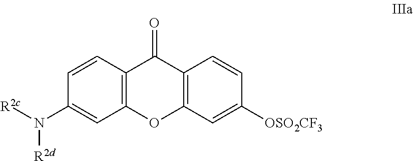

which intermediate is then reacted with at least one equivalent of a compound of formula IVb; or
(ii) reacted with at least one equivalent of a compound of formula IVb to provide the intermediate of formula IIIb

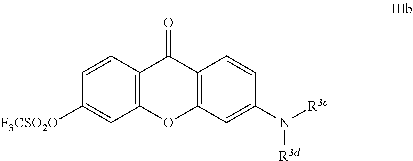

which intermediate is then reacted with at least one equivalent of a compound of formula IVb.

In any event, the reaction between the compound of formula III (or IIIa or IIIb) and the compounds of formulae IVa and IVb may take place at, for example, ambient or elevated temperature (e.g. 70 to 110° C., such as 90° C.) in the presence of a suitable organic solvent (e.g. a polar, aprotic solvent such as dimethylsulfoxide). Further, the reaction may use anywhere from 1 to 7 equivalents (e.g. 5 equivalents) each of compounds of formulae IVa and IVb.

The process may be conducted so as to prepare a compound of formula V, IIIa or IIIb as a final product. Thus, according to further aspects of the invention, there is provided:
(A) a process for the production of a compound of formula V, said process comprising reacting a compound of formula III, as hereinbefore defined with at least one equivalent each of compounds of formulae IVa and IVb, as hereinbefore defined;
(B) a process for the production of a compound of formula V, said process comprising reacting a compound of formula IIIa, as hereinbefore defined with at least one equivalent of a compound of formula IVb, as hereinbefore defined;
(C) a process for the production of a compound of formula V, said process comprising reacting a compound of formula IIIb, as hereinbefore defined with at least one equivalent of a compound of formula IVa, as hereinbefore defined;
(D) a process for the production of a compound of formula IIIa, said process comprising reacting a compound of formula III, as hereinbefore defined with at least one equivalent of a compound of formula IVa, as hereinbefore defined; and
(E) a process for the production of a compound of formula IIIb, said process comprising reacting a compound of formula III, as hereinbefore defined with at least one equivalent of a compound of formula IVb, as hereinbefore defined.

In Step (b) above, the reaction with the Grignard reagent of formula VIa or the lithium reagent of formula VIb may utilise one equivalent of the organometallic reagent and take place at sub-ambient temperature (e.g. from −70 to 10° C., such as 0° C.) in the presence of a suitable aprotic organic solvent (e.g. tetrahydrofuran).

In particular embodiments of the invention, conversion of the intermediate of formula VII to the compound of formula II takes place without isolation of the intermediate (e.g. in the same reaction vessel and solvent as for the formation of that intermediate), for example by addition of aqueous acid (e.g. 2 M hydrochloric acid).

Compounds of formulae I, II, IIIa, IIIb and V may be isolated from their reaction mixtures using conventional techniques.

The compound of formula III may be obtained by methods known to those skilled in the art, such as those described in Chang et al., J. Am. Chem. Soc. 2005, 127, 16652-16659 and Wu et al., Org. Lett. 2008, 10, 1779-1782.

The above-described process for preparing compounds of formula II has the advantage that it may allow for a convenient, high-yielding and scalable preparation of those compounds from readily available precursors.

Further, the process for preparing compounds of formula II may also have the advantage that the compound of formula II is produced in higher yield, in higher purity, in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art.

Certain intermediate compounds described herein are novel. Thus, according to further aspects of the invention, there is provided:
(i) a compound of formula IIIa, as hereinbefore defined (e.g. a compound of formula IIIa in which R^{2c} and R^{2d} together take the same definition as R^{2a} and R^{2b}, as hereinbefore defined); and
(ii) a compound of formula IIIb, as hereinbefore defined (e.g. a compound of formula IIIb in which R^{3c} and R^{3d} together take the same definition as R^{3a} and R^{3b}, as hereinbefore defined).

### FIGURES

The invention will now be described with reference to the following none limiting figures and examples.

All references herein mentioned are hereby incorporated by reference.

FIG. 1 shows the intracellular localization of compound 11 in HSC2 cells;

FIG. 2 shows histograms and mean percentage of annexin V-FITC binding to PS as an indicator of apoptosis in HSC2 cells treated with compound 11;

FIG. 3 shows the effects of compound 11 on cell cycle; and

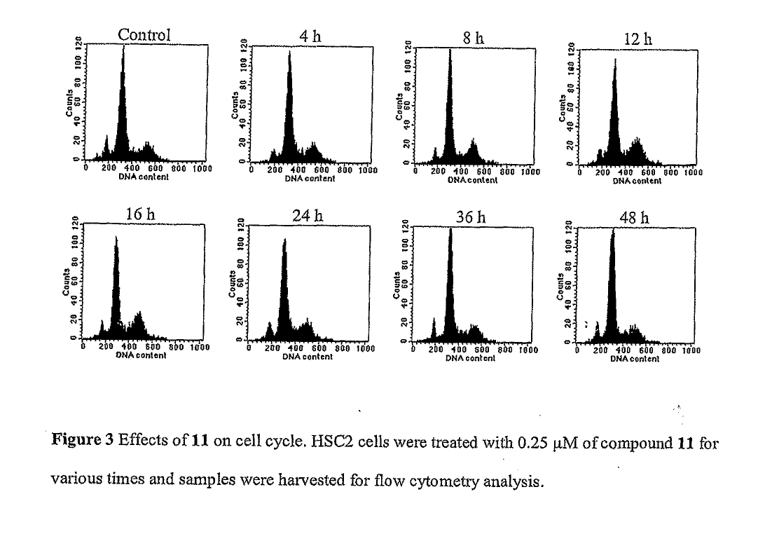

FIG. 4 shows a comparison between the general formulae of rosamines and rhodamines.

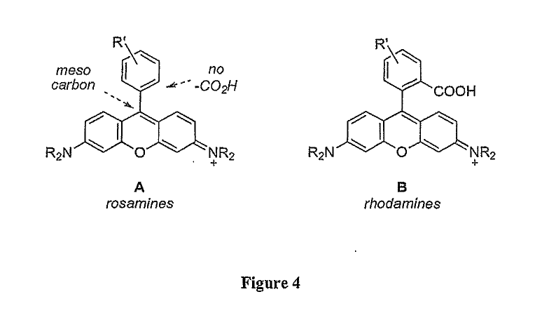

### DETAILED DESCRIPTION OF EMBODIMENTS OF THE INVENTION

### General Experimental Methods

All reactions were carried out under an atmosphere of dry nitrogen. Glassware was oven-dried prior to use. Unless otherwise indicated, common reagents or materials were obtained from commercial source and used without further purification. All the solvents were used after appropriate distillation or purification.

Flash column chromatography was performed using silica gel 60 (230-400 mesh). Analytical thin layer chromatography (TLC) was carried out on Merck silica gel plates with QF-254 indicator and visualized by UV. Fluorescence spectra were obtained on a Varian Cary Eclipse fluorescence spectrophotometer at room temperature. Absorption spectra were obtained on a Varian 100 Bio UV-Vis spectrophotometer at room temperature. IR spectra were recorded on a Bruker Tensor 27 spectrometer.

¹H and ¹³C spectra were recorded on a Varian 300 (300 MHz ¹H; 75 MHz ¹³C) or Varian 500 (500 MHz ¹H; 125 MHz ¹³C) spectrometer at room temperature. Chemical shifts were reported in ppm relative to the residual CDCl₃ (δ 7.24 ppm ¹H; δ 77.0 ppm ¹³C), CD₃OD (δ 3.31 ppm ¹H; δ 49.0 ppm ¹³C) or d⁶-DMSO (δ 2.49 ppm ¹H; δ 39.5 ppm ¹³C). ¹⁹F NMR were acquired on a Varian 300 (300 MHz ¹H; 282 MHz ¹⁹F) spectrometer. CFCl₃ was used as an external reference for the ¹⁹F NMR spectra. Coupling constants (J) were reported in Hertz.

### Photophysical Properties and Determination of Quantum Yields

Steady-state fluorescence spectroscopic studies were performed on a Cary Eclipse fluorometer. The slit width was 5 nm for both excitation and emission. The relative quantum yields of the samples were obtained by comparing the area under the corrected emission spectrum of the test sample with that of a standard. The quantum efficiencies of fluorescence were obtained from multiple measurements (N=3) with the following equation:

Φₓ=Φₛₜ(Iₓ/Iₛₜ)(Aₛₜ/Aₓ)(ηₓ²/ηₛₜ²)

Where Φₛₜ is the reported quantum yield of the standard, I is the area under the emission spectra, A is the absorption at the excitation wavelength and η is the refractive index of the solvent used, measured on a pocket refractometer from ATAGO. X subscript denotes test sample, and st denotes standard.

### Example 1a

### General Procedure for Preparation of Symmetrical Compounds of Formula V

### (a) Synthesis of 3,6-Bis(trifluoromethanesulfonyloxy)-xanthen-9-one

3,6-Dihydroxy-xanthen-9-one² (6.85 g, 30 mmol) was dissolved in 150 mL CH₂Cl₂ and pyridine (24.5 mL, 300 mmol) was added slowly over 5 min at 0° C. The mixture was stirred at 0° C. for 10 min then Tf₂O (15 mL, 90 mmol) was added dropwise over 10 min. The reaction mixture was warmed to room temperature slowly and stirred for 24 h. The reaction was quenched with water and the organic layer was washed with water (1×30 mL), 1N HCl (3×30 mL), brine (1×30 mL) and dried over Na₂SO₄. The solvents were removed under reduced pressure and the residue was recrystallized from CH₂Cl₂/hexanes to afford the pure product as a white crystal (13.2 g, 89%). ¹H NMR (500 MHz, CDCl₃) δ 8.43 (d, 2H, J=8.9 Hz), 7.47 (d, 2H, J=2.3 Hz), 7.33 (dd, 2H, J=8.9, 2.3 Hz); ¹³C NMR (125 MHz, CDCl₃) δ 174.5, 156.6, 153.3, 129.6, 121.4, 118.7 (q, J=319.1 Hz), 118.2, 111.4; ¹⁹F NMR (282 MHz, CDCl₃) δ 104.2 (s).

### (b) Synthesis of compounds of symmetrical formula V

3,6-Bis(trifluoromethanesulfonyloxy)-xanthen-9-one (1.0 eq; see step (a) above) was dissolved in DMSO (0.2 M) and the appropriate amine (10 eq) was added. The reaction mixture was heated to 90° C. and stirred for 12 h. After cooling to room temperature, the reaction was quenched with water. The precipitate was collected, washed with saturated Na₂CO_{3 (aq.)} and water to give the crude product, which was recrystallized from EtOAc/Hexanes to afford the pure product.

### (i) 3,6-Di-piperidin-1-yl-xanthen-9-one

Yellow solid (3.0 g, 89%). ¹H NMR (300 MHz, d⁶-DMSO) δ 7.86 (d, 2H, J=9.0 Hz), 6.98 (dd, 2H, J=9.0, 2.3 Hz), 6.74 (d, 2H, J=2.3 Hz), 3.41 (br, 8H), 1.60 (br, 12H); ¹³C NMR (75 MHz, d⁶-DMSO) δ 173.0, 157.7, 154.8, 126.8, 111.7, 111.3, 98.8, 47.8, 24.8, 23.9. MS (ESI) m/z calcd for (M+H)⁺ C₂₃H₂₆N₂O₂ 363.21. found 363.21.

### (ii) 3,6-Di-morpholin-4-yl-xanthen-9-one

White solid (308 mg, 93%). ¹H NMR (500 MHz, CDCl₃) δ 8.14 (d, 2H, J=9.0 Hz), 6.86 (dd, 2H, J=9.0, 2.4 Hz), 6.67 (d, 2H, J=2.4 Hz), 3.86 (t, 8H, J=4.9 Hz), 3.33 (t, 8H, J=4.9 Hz); ¹³C NMR (125 MHz, CDCl₃) δ 175.1, 158.0, 155.3, 127.7, 114.1, 111.2, 100.0, 66.5, 47.5. MS (ESI) m/z calcd for (M+H)⁺ C₂₁H₂₃N₂O₄ 367.17. found 367.17.

### (iii) 3,6-Bis-(4-Boc-piperazin-1-yl)-xanthen-9-one

White solid (680 mg, 60%). ¹H NMR (500 MHz, CDCl₃) δ 8.13 (d, 2H, J=9.0 Hz), 6.86 (dd, 2H, J=9.0, 2.3 Hz), 6.66 (d, 2H, J=2.3 Hz), 3.59 (t, 8H, J=5.2 Hz), 3.37 (t, 8H, J=5.2 Hz), 1.47 (s, 18H); ¹³C NMR (125 MHz, CDCl₃) δ 175.0, 158.1, 155.0, 154.6, 127.8, 114.0, 111.7, 100.3, 80.2, 47.3 (2C), 28.4. MS (ESI) m/z calcd for (M+H)⁺ C₃₁H₄₁N₄O₆ 565.30. found 565.29.

### (iv) 3,6-Di-pyrrolidin-1-yl-xanthen-9-one

Yellow solid (90 mg, 18%). ¹H NMR (500 MHz, CDCl₃) δ 8.08 (d, 2H, J=8.9 Hz), 6.50 (dd, 2H, J=8.9, 2.2 Hz), 6.28 (d, 2H, J=2.2 Hz), 3.34 (t, 8H, J=6.6 Hz), 2.01 (t, 8H, J=6.6 Hz); ¹³C NMR (125 MHz, CDCl₃) δ 175.0, 158.1, 151.6, 127.6, 111.5, 109.1, 96.5, 47.6, 25.4. MS (ESI) m/z calcd for (M+H)⁺ C₂₁H₂₃N₂O₂ 335.18. found 335.18.

### (v) Dimethyl 1,1′-(9-oxo-9H-xanthene-3,6-diyl)dipiperidine-4-carboxylate

Light yellow solid (500 mg, 51%). ¹H NMR (500 MHz, CDCl₃) δ 8.09 (d, 2H, J=8.9 Hz), 6.85 (dd, 2H, J=8.9, 2.3 Hz), 6.65 (d, 2H, J=2.3 Hz), 3.87-3.83 (m, 4H), 3.69 (s, 6H), 3.03-2.98 (m, 4H), 2.58-2.52 (m, 2H), 2.04-2.00 (m, 4H), 1.87-1.79 (m, 4H); ¹³C NMR (125 MHz, CDCl₃) δ 175.0, 174.8, 158.1, 154.9, 127.7, 113.5, 111.6, 100.1, 51.8, 47.2, 40.7, 27.5. MS (ESI) m/z calcd for (M+H)⁺ C₂₇H₃₁N₂O₆ 479.22. found 479.23.

### Example 1b

### Procedure for Preparation of Asymmetric Compounds of Formula V

### (a) Synthesis of intermediate 3-(trifluoromethanesulfonyloxy)-6-(piperidin-1-yl)-xanthen-9-one

3,6-Bis(trifluoromethanesulfonyloxy)-xanthen-9-one (492 mg, 1.0 mmol; see Example 1a, step (a) above) was dissolved in 10 mL DMSO and piperidine (0.49 mL, 5.0 mmol) was added. After stirring at 25° C. for 1.5 h, the solution was diluted with 30 mL CH₂Cl₂ and washed with water (3×30 mL). The solvents were removed under reduced pressure and the residue was purified by flash chromatography (10% to 20% EtOAc/Hexanes) to afford the pure product (372 mg, 87%) as a light yellow solid. R_{f}=0.66 (40% EtOAc/hexanes). ¹H NMR (300 MHz, d⁶-DMSO) δ 8.25 (d, 1H, J=8.8 Hz), 7.91 (d, 1H, J=9.1 Hz), 7.79 (d, 1H, J=2.4 Hz), 7.50 (dd, 1H, J=8.8, 2.4 Hz), 7.07 (dd, 1H, J=9.1, 2.4 Hz), 6.82 (d, 1H, J=2.4 Hz), 3.50-3.47 (m, 4H), 1.60 (br, 6H); ¹³C NMR (75 MHz, d⁶-DMSO) δ 172.7, 158.2, 155.9, 155.3, 151.9, 128.5, 127.3, 121.8, 118.4 (q, J=320.5 Hz), 117.1, 112.1, 111.3, 110.8, 98.2, 47.7, 24.9, 23.9. MS (ESI) m/z calcd for (M+H)⁺ C₁₉H₁₇F₃NO₅S 428.08. found 428.08.

### (b) Synthesis of 3-(piperidin-1-yl)-6-(morpholin-4-yl)-xanthen-9-one

Morpholine (0.66 mL, 7.5 mmol) was added to the solution of 3-(trifluoromethanesulfonyloxy)-6-(piperidin-1-yl)-xanthen-9-one (321 mg, 0.75 mmol; see step (a) above) in 10 mL DMSO. The reaction mixture was heated to 90° C. and stirred for 12 h. After cooling to room temperature, the solution was diluted with 30 mL CH₂Cl₂ and washed with saturated Na₂CO_{3 (aq.)} (1×30 mL), water (1×30 mL) and dried over Na₂SO₄. The solvents were removed under reduced pressure and the residue was purified by flash chromatography (40% EtOAc/Hexanes) to afford the pure product 11 (261 mg, 96%) as a light yellow solid. R_{f}=0.20 (40% EtOAc/hexanes). ¹H NMR (500 MHz, CDCl₃) δ 8.13 (d, 1H, J=9.1 Hz), 8.09 (d, 1H, J=9.1 Hz), 6.86-6.83 (m, 2H), 6.66 (d, 1H, J=2.2 Hz), 6.64 (d, 1H, J=2.2 Hz), 3.85 (t, 4H, J=4.9 Hz), 3.39-3.37 (m, 4H), 3.32 (t, 4H, J=4.9 Hz), 1.66 (br, 6H); ¹³C NMR (125 MHz, CDCl₃) δ 175.0, 158.3, 158.0, 155.4, 155.1, 127.7, 127.6, 114.3, 112.8, 111.5, 111.1, 100.1, 99.4, 66.5, 48.6, 47.6, 25.3, 24.3. MS (ESI) m/z calcd for (M+H)⁺ C₂₂H₂₅N₂O₃ 365.19. found 365.17.

### Example 2a

### General Procedure for Preparation of Compounds of Formula I

An appropriate Grignard reagent or lithium reagent (1.0 mmol) was added dropwise over 1 min to the solution of 3,6-diamino-xanthen-9-one (0.2 mmol; see Example 1 above) in 5 mL THF at 0° C. After stirring for 12 h at room temperature, the reaction mixture was quenched by addition of 2 mL 2M HCl_{(aq.)} and stirred for 10 min then diluted with 20 mL CH₂Cl₂. The organic layer was washed with water and brine, dried over Na₂SO₄, and concentrated under reduced pressure. The residue was purified by flash chromatography (5% to 10% MeOH/CH₂Cl₂) to give the pure product.

### (i) Compound 7 (Table 1)

Purple solid (71 mg, 89%). R_{f}=0.28 (10% MeOH/CH₂Cl₂). ¹H NMR (500 MHz, CDCl₃) δ 8.01 (d, 2H, J=9.6 Hz), 7.22 (dd, 2H, J=9.6, 2.5 Hz), 6.77 (d, 2H, J=2.5 Hz), 3.66 (br, 8H), 2.87 (s, 3H), 1.72 (br, 12H); ¹³C NMR (125 MHz, CDCl₃) δ 157.6, 156.4, 156.3, 130.1, 114.8, 114.0, 96.8, 48.8, 25.8, 24.1, 14.8; IR (thin film) 1643, 1597, 1486, 1401, 1235, 1200 cm⁻¹; HRMS (ESI) m/z calcd for (M-Cl)⁺ C₂₄H₂₉N₂O 361.2280. found 361.2287.

### (ii) Compound 4 (Table 1)

Green solid (94 mg, 98%). R_{f}=0.26 (10% MeOH/CH₂Cl₂). ¹H NMR (500 MHz, CD₃OD) δ 7.59-7.46 (m, 3H), 7.30-7.23 (m, 7H), 3.87-3.85 (m, 8H), 3.79-3.77 (m, 8H), 2.06 (s, 3H); ¹³C NMR (125 MHz, CD₃OD) δ 160.2, 159.9, 159.1, 137.2, 133.0, 132.8, 131.9, 131.4, 130.1, 127.3, 116.3, 115.8, 98.6, 67.4, 48.5, 19.6; IR (thin film) 1646, 1590, 1481, 1415, 1383, 1235, 1190 cm⁻¹; HRMS (ESI) m/z calcd for (M-Cl)⁺ C₂₈H₂₉N₂O₃ 441.2178. found 441.2184.

(iii) Compound 1 (Table 1)

Green solid (47 mg, 53%). R_{f}=0.28 (10% MeOH/CH₂Cl₂). ¹H NMR (500 MHz, CD₃OD) δ 7.58-7.45 (m, 3H), 7.26 (d, 1H, J=7.5 Hz), 7.16 (d, 2H, J=9.5 Hz), 6.95 (dd, 2H, J=9.5, 2.2 Hz), 6.84 (d, 2H, J=2.2 Hz), 3.63 (br, 8H), 2.15 (br, 8H), 2.06 (s, 3H); ¹³C NMR (125 MHz, CD₃OD) δ 159.3, 159.1, 156.4, 137.2, 133.4, 132.4, 131.9, 131.2, 130.1, 127.3, 116.6, 114.7, 97.9, 50.1, 26.3, 19.6. IR (thin film) 1648, 1596, 1413, 1378, 1344, 1189 cm⁻¹; HRMS (ESI) m/z calcd for (M-Cl)⁺ C₂₈H₂₉N₂O 409.2280. found 409.2277.

### (iv) Compound 3 (Table 1)

Green solid (93 mg, 98%). R_{f}=0.30 (10% MeOH/CH₂Cl₂). ¹H NMR (500 MHz, CD₃OD) δ 7.58-7.45 (m, 3H), 7.29-7.19 (m, 7H), 3.86-3.81 (m, 8H), 3.74-3.72 (m, 4H), 2.07 (s, 3H), 1.85-1.75 (m, 6H); ¹³C NMR (125 MHz, CD₃OD) δ 160.3, 159.5, 159.3, 158.7, 158.6, 137.3, 133.1, 133.0, 132.5, 131.9, 131.3, 130.1, 127.3, 116.8, 115.7, 115.6, 115.3, 98.7, 98.1, 67.4, 50.2, 48.4, 27.3, 25.3, 19.6; IR (thin film) 1646, 1590, 1480, 1415, 1387, 1236, 1189 cm⁻¹; HRMS (ESI) m/z calcd for (M-Cl)⁺ C₂₉H₃₁N₂O₂ 439.2386. found 439.2384.

### (v) Compound 8 (Table 1)

Purple solid (66 mg, 70%). R_{f}=0.34 (10% MeOH/CH₂Cl₂). ¹H NMR (500 MHz, CDCl₃) δ 7.99 (d, 2H, J=9.7 Hz), 7.23-7.15 (m, 7H), 6.87 (d, 2H, J=2.5 Hz), 4.68 (s, 2H), 3.70 (br, 8H), 1.74 (br, 12H); ¹³C NMR (125 MHz, CDCl₃) δ 158.1, 156.5, 156.3, 137.1, 130.3, 129.1, 128.1, 127.2, 115.2, 113.9, 97.2, 49.0, 33.3, 25.9, 24.1; IR (thin film) 1643, 1594, 1480, 1424, 1397, 1237, 1169 cm⁻¹; HRMS (ESI) m/z calcd for (M-Cl)⁺ C₃₀H₃₃N₂O 437.2593. found 437.2595.

### (vi) Compound 9 (Table 1)

Green solid (92 mg, 100%). R_{f}=0.35 (10% MeOH/CH₂Cl₂). ¹H NMR (500 MHz, CDCl₃) δ 7.46-7.45 (m, 3H), 7.21-7.18 (m, 4H), 6.97 (dd, 2H, J=9.6, 2.5 Hz), 6.79 (d, 2H, J=2.5 Hz), 3.60 (br, 8H), 1.62 (br, 12H); ¹³C NMR (125 MHz, CDCl₃) δ 157.9, 156.4, 156.0, 131.7, 131.3, 130.0, 129.0, 128.6, 114.5, 113.2, 96.9, 48.8, 25.6, 23.7; IR (thin film) 1646, 1592, 1483, 1414, 1391, 1235, 1190 cm⁻¹; HRMS (ESI) m/z calcd for (M-Cl)⁺ C₂₉H₃₁N₂O 423.2436. found 423.2437.

(vii) Compound 11 (Table 1)

Green solid (105 mg, 90%) [Made from 4-Iodophenyl magnesium chloride, which was prepared from 1,4-di-iodobenzene with ⁱPrMgCl]. R_{f}=0.36 (10% MeOH/CH₂Cl₂). ¹H NMR (500 MHz, CDCl₃) δ 7.95 (d, 2H, J=8.4 Hz), 7.27 (d, 2H, J=9.7 Hz), 7.12 (d, 2H, J=8.4 Hz), 7.07 (dd, 2H, J=9.7, 2.5 Hz), 6.99 (d, 2H, J=2.5 Hz), 3.73 (br, 8H), 1.76 (br, 12H); ¹³C NMR (125 MHz, CDCl₃) δ 158.0, 156.2, 155.0, 138.0, 131.5, 131.0, 130.9, 114.9, 113.1, 97.3, 96.7, 49.1, 25.8, 24.0; IR (thin film) 1644, 1592, 1483, 1420, 1391, 1235, 1193 cm⁻¹; HRMS (ESI) m/z calcd for (M-Cl)⁺ C₂₉H₃₀IN₂O 549.1403. found 549.1399. Anal. Calcd for C₂₉H₃₀Cl₁N₂O: C, 59.55; H, 5.17; N, 4.79. Found: C, 58.00; H, 5.17; N, 4.71. The elemental analysis data are consistent with the presence of one molecule of water per molecule of product.

(viii) Compound 12 (Table 1)

Green solid (98 mg, 100%). R_{f}=0.33 (10% MeOH/CH₂Cl₂). ¹H NMR (500 MHz, CDCl₃) δ 7.38 (d, 2H, J=9.6 Hz), 7.27 (d, 2H, J=8.8 Hz), 7.08 (d, 2H, J=8.8 Hz), 7.04 (dd, 2H, J=9.6, 2.5 Hz), 6.88 (d, 2H, J=2.5 Hz), 3.87 (s, 3H), 3.67 (br, 8H), 1.71 (br, 12H); ¹³C NMR (125 MHz, CDCl₃) δ 161.3, 158.2, 157.0, 156.2, 132.1, 131.2, 123.5, 114.6, 114.4, 113.5, 97.3, 55.5, 48.9, 25.8, 24.0; IR (thin film) 1644, 1592, 1480, 1391, 1235, 1191 cm⁻¹; HRMS (ESI) m/z calcd for (M-Cl)⁺ C₃₀H₃₃N₂O₂ 453.2542. found 453.2546.

### (ix) Compound 13 (Table 1)

Green solid (97 mg, 100%). R_{f}=0.36 (10% MeOH/CH₂Cl₂). ¹H NMR (500 MHz, CDCl₃) δ 7.50-7.47 (m, 1H), 7.15 (d, 2H, J=9.5 Hz), 7.08-7.04 (m, 3H), 6.99 (dd, 2H, J=9.5, 2.5 Hz), 6.83 (d, 2H, J=2.5 Hz), 3.65-3.63 (m, 11H), 1.67 (br, 12H); ¹³C NMR (125 MHz, CDCl₃) δ 158.3, 156.5, 156.4, 155.2, 132.0 (2C), 130.5, 120.8, 120.4, 114.6, 114.1, 111.6, 97.2, 55.8, 49.1, 25.9, 24.1; IR (thin film) 1646, 1590, 1480, 1415, 1390, 1233, 1187 cm⁻¹; HRMS (ESI) m/z calcd for (M-Cl)⁺ C₃₀H₃₃N₂O₂ 453.2542. found 453.2540.

### (x) Compound 10 (Table 1)

Green solid (69 mg, 74%). R_{f}=0.25 (10% MeOH/CH₂Cl₂). ¹H NMR (500 MHz, CDCl₃) δ 7.74 (dd, 1H, J=4.5, 1.7 Hz), 7.63 (d, 2H, J=9.5 Hz), 7.32-7.30 (m, 2H), 7.12 (dd, 2H, J=9.5, 2.5 Hz), 6.92 (d, 2H, J=2.5 Hz), 3.72 (br, 8H), 1.74 (br, 12H); ¹³C NMR (125 MHz, CDCl₃) δ 158.0, 156.3, 149.4, 132.1, 131.9, 130.9, 130.4, 128.3, 114.9, 113.9, 97.4, 49.1, 25.9, 24.1; IR (thin film) 1642, 1592, 1482, 1415, 1391, 1237, 1192 cm⁻¹; HRMS (ESI) m/z calcd for (M-Cl)⁺ C₂₇H₂₉N₂OS 429.2001. found 429.1995.

### (xi) Compound 14 (Table 1)

Green solid (100 mg, 96%). R_{f}=0.32 (10% MeOH/CH₂Cl₂). [Made from 2,6-dimethoxyphenyl lithium, which was prepared from 1,3-dimethoxybenzene with ⁿBuLi]. ¹H NMR (500 MHz, CDCl₃) δ 7.49 (t, 1H, J=8.5 Hz), 7.18 (d, 2H, J=9.5 Hz), 7.01 (dd, 2H, J=9.5, 2.5 Hz), 6.88 (d, 2H, J=2.5 Hz), 6.71 (d, 2H, J=8.5 Hz), 3.69-3.67 (m, 8H), 3.62 (s, 6H), 1.73 (br, 12H); ¹³C NMR (125 MHz, CDCl₃) δ 158.3, 157.4, 156.5, 153.7, 132.3, 131.7, 114.6, 114.5, 108.4, 104.0, 97.0, 55.9, 48.9, 25.8, 24.1; HRMS (ESI) m/z calcd for (M-Cl)⁺ C₃₁H₃₅N₂O₃ 483.2648; found 483.2656.

(xii) Compound 2 (Table 1)

Green solid (95 mg, 99%). R_{f}=0.32 (10% MeOH/CH₂Cl₂). ¹H NMR (500 MHz, CD₃OD) δ 7.58-7.44 (m, 3H), 7.26-7.16 (m, 7H), 3.79 (br, 8H), 2.07 (s, 3H), 1.82-1.76 (m, 12H); ¹³C NMR (125 MHz, CD₃OD) δ 159.9, 158.6, 158.2, 137.2, 133.2, 132.7, 131.9, 131.3, 130.1, 127.3, 116.1, 115.0, 98.2, 50.0, 27.1, 25.3, 19.6; IR (thin film) 1646, 1588, 1482, 1414, 1389, 1233, 1187 cm⁻¹; HRMS (ESI) m/z calcd for (M-Cl)⁺ C₃₀H₃₃N₂O 437.2593. found 437.2598.

(xiii) Compound 5 (Table 1)

Purple solid (121 mg, 90%). R_{f}=0.37 (10% MeOH/CH₂Cl₂). ¹H NMR (500 MHz, CD₃OD) δ 7.60-7.46 (m, 3H), 7.29-7.22 (m, 7H), 3.84 (br, 8H), 3.67 (br, 8H), 2.06 (s, 3H), 1.50 (s, 18H); ¹³C NMR (125 MHz, CD₃OD) δ 160.2, 159.9, 158.8, 156.2, 137.2, 133.0, 132.8, 131.9, 131.4, 130.1, 127.3, 116.4, 115.7, 98.6, 81.9, 47.8 (2C), 28.6, 19.6; IR (thin film) 1693, 1646, 1591, 1480, 1413, 1388, 1227, 1161 cm⁻¹; HRMS (ESI) m/z calcd for (M-Cl)⁺ C₃₈H₄₇N₄O₅ 639.3546. found 639.3553.

(xiv)

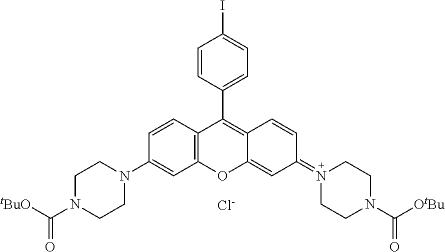

Green solid (107 mg, 68%). R_{f}=0.31 (10% MeOH/CH₂Cl₂). ¹H NMR (500 MHz, CDCl₃) δ 7.94 (d, 2H, J=8.3 Hz), 7.37 (d, 2H, J=9.5 Hz), 7.19-7.15 (m, 4H), 7.11 (d, 2H, J=8.3 Hz), 3.80 (br, 8H), 3.67-3.65 (m, 8H), 1.45 (s, 18H); ¹³C NMR (125 MHz, CDCl₃) δ 158.2, 156.9, 156.8, 154.4, 138.2, 131.8, 131.0, 130.8, 115.4, 114.0, 98.3, 97.3, 80.7, 47.2 (2C), 28.3; IR (thin film) 1694, 1644, 1592, 1479, 1415, 1387, 1226, 1161 cm⁻¹; HRMS (ESI) m/z calcd for (M-Cl)⁺ C₃₇H₄₄N₄O₅ 751.2356. found 751.2342.

(xv)

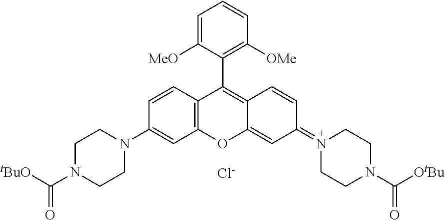

Purple solid (135 mg, 94%). R_{f}=0.31 (10% MeOH/CH₂Cl₂). ¹H NMR (500 MHz, CDCl₃) δ 7.50 (t, 1H, J=8.5 Hz), 7.29 (d, 2H, J=9.4 Hz), 7.20 (dd, 2H, J=9.4, 2.5 Hz), 7.12 (d, 2H, J=2.5 Hz), 6.71 (d, 2H, J=8.5 Hz), 3.77 (br, 8H), 3.65-3.63 (m, 8H), 3.62 (s, 6H), 1.44 (s, 18H); ¹³C NMR (125 MHz, CDCl₃) δ 158.3, 157.4, 157.0, 155.4, 154.5, 132.5, 132.0, 115.5, 115.0, 108.3, 104.1, 97.8, 80.6, 55.9, 47.0 (2C), 28.3. HRMS (ESI) m/z calcd for (M-Cl)⁺ C₃₉H₄₉N₄O₇ 685.3601. found 685.3598.

### Example 2b

### Preparation of Compounds of Formula I from Other Compounds of Formula I

(i)

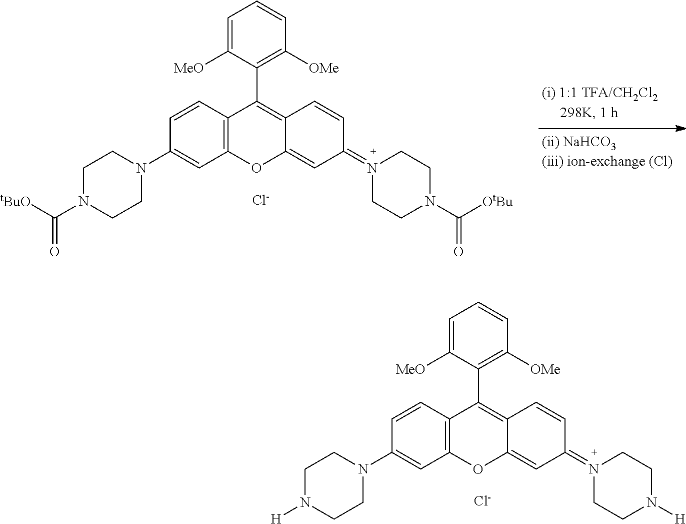

A solution of the rosamine of Example 2a(xv) (50 mg, 0.07 mmol) in 5 mL TFA/CH₂Cl₂ (1:1) was stirred at room temperature for 1 h. The solvents were removed with a N₂ stream. The residue was dissolved in 15 mL ⁱPrOH/CH₂Cl₂ (1:1) and washed with saturated NaHCO_{3 (aq.)}, water, and brine then dried over Na₂SO₄. The solvents were removed under reduced pressure. The residue was dissolved in 10 mL MeOH and 0.5 g Amberlite IRA-400 (Cl) ion exchange resin was added. The mixture was stirred at room temperature for 1 h and filtered through celite. The solvent was removed under reduced pressure. The ion-exchange process was repeated twice. The crude product was purified by reverse phase MPLC (H₂O—50% CH₃CN/H₂O) to give the pure product (30 mg, 83%) as a purple solid. ¹H NMR (500 MHz, CD₃OD) δ 7.66 (t, 1H, J=8.6 Hz), 7.44 (d, 2H, J=9.3 Hz), 7.37 (dd, 2H, J=9.3, 2.2 Hz), 7.35 (d, 2H, J=2.2 Hz), 6.94 (d, 2H, J=8.6 Hz), 4.10 (t, 8H, J=5.3 Hz), 3.67 (s, 6H), 3.46 (t, 8H, J=5.3 Hz); ¹³C NMR (125 MHz, CD₃OD) δ 160.0, 158.9, 158.6, 158.5, 134.3, 133.4, 117.1, 116.7, 109.4, 105.5, 99.3, 56.6, 45.2, 44.1; HRMS (ESI) m/z calcd for (M-Cl)⁺ C₂₉H₃₃N₄O₃ 485.2553. found 485.2557.

(ii)

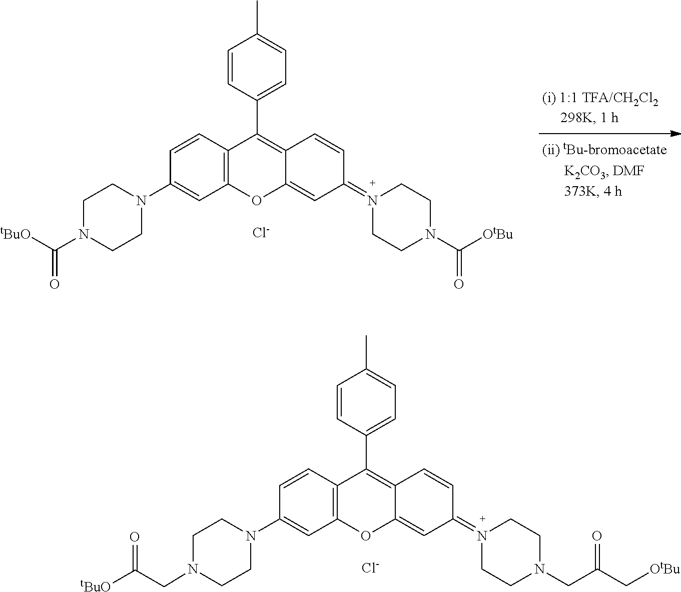

The rosamine of Example 2a(xiv) (39 mg, 0.05 mmol) was dissolved in 2 mL TFA/CH₂Cl₂ (1:1). The solution was stirred at 25° C. for 1 h. The solvents were removed with nitrogen stream. The residue was dissolved in 2 mL DMF then K₂CO₃ (69 mg, 0.5 mmol) and tert-butyl bromoacetate (74 μL, 0.5 mmol) were added. The mixture was heated to 100° C. and stirred for 4 h. After cooling to room temperature, the mixture was diluted with CH₂Cl₂ and washed with water, dried over Na₂SO₄ and concentrated under reduced pressure. The residue was purified by flash chromatography (10% MeOH/CH₂Cl₂, R_{f}=0.28) to afford the pure product (31 mg, 76%) as a purple solid. ¹H NMR (500 MHz, CDCl₃) δ 7.93 (d, 2H, J=8.4 Hz), 7.29 (d, 2H, J=9.4 Hz), 7.10-7.06 (m, 6H), 3.79 (br, 8H), 3.20 (s, 4H), 2.79 (br, 8H), 1.43 (s, 18H); ¹³C NMR (125 MHz, CDCl₃) δ 169.8, 158.2, 156.8, 156.1, 138.2, 131.6, 131.1, 131.0, 115.0, 113.8, 98.1, 97.0, 82.1, 59.3, 52.2, 47.1, 28.0; IR (thin film) 1735, 1688, 1644, 1594, 1482, 1391, 1236, 1195, 1152 cm⁻¹; HRMS (ESI) m/z calcd for (M-Cl)⁺ C₃₉H₄₈IN₄O₅ 779.2669. found 779.2671.

(iii)

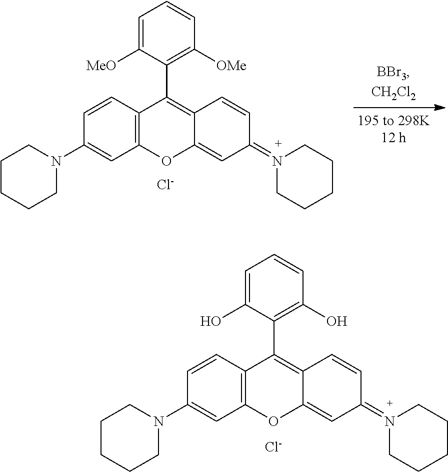

BBr₃ (0.19 mL, 2.0 mmol) was added dropwise over 1 min to the solution of the rosamine of Example 2a(xi) (compound 14 of Table 1) (104 mg, 0.2 mmol) in 4 mL CH₂Cl₂ at −78° C. The solution was warmed to room temperature slowly and stirred for 12 h. The reaction was quenched with ice-water and the mixture was extracted with 1:1 ⁱPrOH/CH₂Cl₂ (3×15 mL). The organic layer was washed with water (1×20 mL), brine (1×20 mL), dried over Na₂SO₄ and concentrated under reduced pressure. The residue was dissolved in 10 mL MeOH and 0.5 g Amberlite IRA-400 (Cl) ion exchange resin was added. The mixture was stirred at room temperature for 1 h and filtered through celite. The solvent was removed under reduced pressure. The ion-exchange process was repeated twice. The crude product was purified by flash chromatography (5% to 10% MeOH/CH₂Cl₂) to afford the product (85 mg, 87%) as a green solid. R_{f}=0.15 (10% MeOH/CH₂Cl₂). ¹H NMR (500 MHz, CDCl₃/CD₃OD 1:1) δ 7.44 (d, 2H, J=9.6 Hz), 7.23 (t, 1H, J=8.3 Hz), 7.07 (dd, 2H, J=9.6, 2.6 Hz), 6.94 (d, 2H, J=2.6 Hz), 6.54 (d, 2H, J=8.3 Hz), 3.71-3.69 (m, 8H), 1.79-1.74 (m, 12H); ¹³C NMR (125 MHz, CDCl₃/CD₃OD 1:1) δ 159.3, 157.4, 156.4, 156.1, 133.2, 132.5, 115.5, 114.9, 107.4, 107.3, 97.5, 49.4, 26.5, 24.8; HRMS (ESI) m/z calcd for (M-Cl)⁺ C₂₉H₃₁N₂O₃ 455.2335. found 455.2340.

(iv)

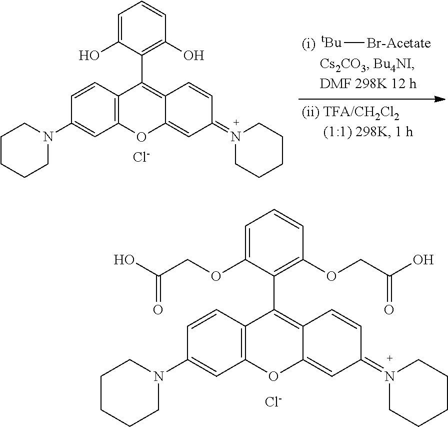

The rosamine of Example 2b(iii) above (Compound 15 of Table 1) (137 mg, 0.28 mmol), Cs₂CO₃ (456 mg, 1.4 mmol), Bu₄NI (310 mg, 0.84 mmol) were dissolved in 5 mL DMF and tert-butyl bromoacetate (0.41 mL, 2.8 mmol) was added. The reaction mixture was stirred at 25° C. for 12 h then diluted with 30 mL CH₂Cl₂, washed with H₂O (3×20 mL), brine (1×20 mL) and dried over Na₂SO₄. The solvents were removed under reduced pressure and the residue was passed through a short pad of silica gel eluting with 5% MeOH/CH₂Cl₂ to give the crude product which was used in the next step without further purification. The crude material was dissolved in 10 mL TFA/CH₂Cl₂ (1:1) and stirred at 25° C. for 1 h. The solvents were removed with a N₂ stream. The residue was dissolved in 30 mL CH₂Cl₂, washed with H₂O (2×20 mL), brine (1×20 mL) and dried over Na₂SO₄. The solvents were removed under reduced pressure. The residue was dissolved in 20 mL MeOH/CH₂Cl₂ (1:1) and 1.0 g Amberlite IRA-400 (Cl) ion exchange resin was added. The mixture was stirred at room temperature for 1 h and filtered through celite. The solvent was removed under reduced pressure. The ion-exchange process was repeated twice. The crude product was purified by reverse phase MPLC(H₂O—60% CH₃CN/H₂O) to afford the pure product as a green solid (128 mg, 75%). ¹H NMR (500 MHz, CDCl₃/CD₃OD 1:1) δ 7.49 (t, 1H, J=8.5 Hz), 7.46 (d, 2H, J=9.6 Hz), 7.05 (dd, 2H, J=9.6, 2.5 Hz), 6.93 (d, 2H, J=2.5 Hz), 6.69 (d, 2H, J=8.5 Hz), 4.52 (s, 4H), 3.72-3.70 (m, 8H), 1.80-1.75 (m, 12H); ¹³C NMR (125 MHz, CDCl₃/CD₃OD 1:1) δ 171.0, 159.2, 157.4, 156.9, 154.0, 133.3, 132.7, 115.4, 114.9, 110.1, 105.7, 97.4, 65.5, 49.4, 26.5, 24.8. HRMS (ESI) m/z calcd for (M-Cl)⁺ C₃₃H₃₅N₂O₇ 571.2444. found 571.2451.

### Example 3

### Biological Properties of the Compounds of Formula I

### Materials and Methods

Materials. ER-Tracker Blue-White DPX, LysoTracker Blue DND-22, rhodamine 123 (Rh123), Sytox Green were purchased from Molecular Probes, Invitrogen (Oregon, USA). Annexin V-PE Apoptosis Detection Kit 1 BD Biosciences (CA, USA). Cell culture reagents were purchased from Gibco, Invitrogen (Auckland, NZ). RNase A, propidium iodide and MTT were purchased from Sigma (St Louis, USA). Cell cultures. HSC2 oral cavity human squamous carcinoma cells were obtained from Health Science Research Resources Bank (Japan). HK1 cell-line is a gift from the University of Hong Kong. Both cell-lines were grown in MEM medium supplemented with 10% FBS. HL-60 human promyelocytic leukemia, MCF-7 breast carcinoma and HCT-116 colon carcinoma cell-lines were obtained from American Tissue Culture Collection (Virginia, USA) and maintained in RPMI 1640 medium supplemented with 10% FBS. OKF6, an immortalized human oral keratinocyte cell-line and NP69, an immortalized human nasopharyngeal epithelial cell-line were obtained from BWH Cell Culture and Microscopy Core at Harvard Institutes of Medicine and the University of Hong Kong respectively, and were maintained in keratinocyte serum-free medium supplemented with epidermal growth factor, bovine pituitary extract and a final Ca²⁺ concentration of 0.3 mM.

In vitro proliferation assay. Approximately 3 000-5 000 (15 000 cells for HL-60) exponentially growing cells were seeded in each well of a 96-well plate with 50 μl of medium and were allowed to adhere overnight. Cells were then treated with each compound at concentrations ranging from 0.01-10 μM giving the final volume of 100 μl in each well. At the end of incubation period, 15 μl of MTT solution (5.0 mg/ml in PBS) was added and incubated for an additional 4 h. Medium and excessive MTT were aspirated and formazan formed was solubilized with 100 μl of DMSO. Absorbance, as a measurement of viable cell number was read at 570 nm with ThermoLabsystems OpsysMR microplate spectrometer. At least three independent experiments were performed and results are presented as an average.

Cellular localization. HSC2 cells grown on round glass coverslips in 12-well plate were co-incubated with 100 nM of compound 11 together with organelle-specific fluorescence probes. The endoplasmic reticulum was labeled with 100 nM of ER-Tracker Blue-White DPX, the lysosomes were stained with 500 nM of LysoTracker Blue DND-22, and the mitochrondria was tracked with 100 nM of Rh123 respectively for 15-30 min of incubation at room temperature. After incubation, cells were gently rinsed in PBS to remove free dyes, and the stained cells were observed using Olympus DSU spinning disk confocal microscope configured with a PlanApo x63 oil objective and iXon EM+(Andor Technology) digital camera. Fluorescent images of X-Y sections at 0.2 μm were collected sequentially using Olympus Cell^{R} software. Organelle-specific fluorescence probes were respectively excited at 365 nm to illuminate ER-tracker and LysoTracker, at 494 nm for Rh123 and at 575 nm for compound 11.

AnnexinV-FITC apoptosis analysis. HSC2 cells grown in 60-mm dishes at 50% confluency were treated with 0.5 μM of compound 11. At various treatment intervals, floating cells in the medium were pooled together with the adherent cells after trypsinization and were washed twice with cold PBS. The cells were resuspended with 1× binding buffer at 1×10⁶ cells/ml. A 100 μl of cell suspension was transferred to a 5 ml tube followed by 5 μA of AnnexinV-FITC and 5 μA of propidium iodide (200 μg/ml in PBS). The cells were gently mixed and incubated for 15 min at RT in the dark before analysed on a FACSCalibur flow cytometer with 488 nm argon laser. The fluorescence data of 10 000 cells were collected with the FL1 detector with 530/30 band pass filter to collect Annexin-FITC fluorescence, and the FL3 detector with a 630 nm long pass filter to collect propidium iodide fluorescence.

Cell cycle analysis. HSC2 cells were treated and collected as above. Cells were then fixed in 70% ice-cold ethanol (v/v in PBS) overnight at 4° C. Following fixation, the cells were washed twice in cold PBS. The pellet was then resuspended in PBS solution containing 20 μg/ml RNase A and 1 μM SYTOX Green for 30 min. The cells were analysed on a FACSCalibur flow cytometer with 488 nm argon laser. The DNA-SYTOX Green fluorescence of 10 000 cells were collected with the FL1 detector with 530/30 band pass filter.

### Results and Discussion

TABLE 1
The structure-activity relationship (SAR) and in vitro cytotoxicity of rosamine analogues in HSC2 cells.

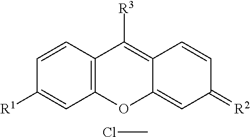

| Compd | R¹ | R² | R³ | IC₅₀ (μM) ± S.D.^{a} in HL60 |
|---|---|---|---|---|
| 1 | 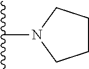 | 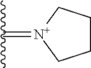 | 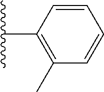 | 0.72 ± 0.09 |
| 2 | 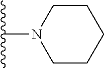 | 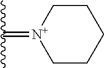 | 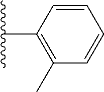 | 0.76 ± 0.03 |
| 3 | 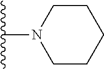 | 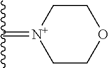 | 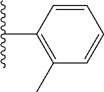 | 0.35 ± 0.01 |
| 4 | 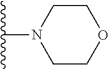 | 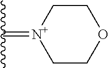 | 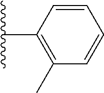 | 8.27 ± 2.16 |
| 5 | 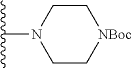 | 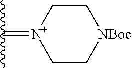 | 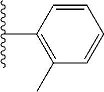 | 0.62 ± 0.07 |
| 6 | 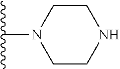 | 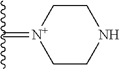 | 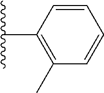 | 53.3 ± 0.3 |
| 7 | 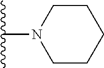 | 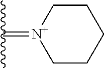 | 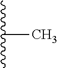 | 3.86 ± 1.46 |
| 8 | 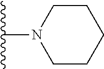 | 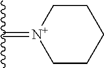 | 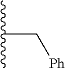 | 0.82 ± 0.04 |
| 9 | 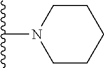 | 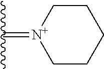 | 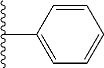 | 0.47 ± 0.13 |
| 10 | 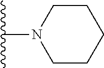 | 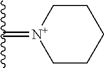 | 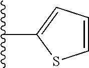 | 0.10 ± 0.04 |
| 11 | 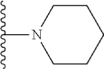 | 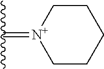 | 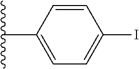 | 0.09 ± 0.01 |
| 12 | 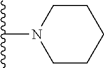 | 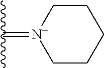 | 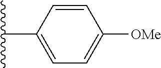 | 0.66 ± 0.17 |
| 13 | 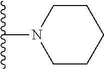 | 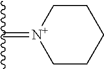 | 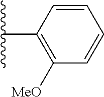 | 0.95 ± 0.01 |
| 14 | 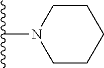 | 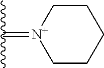 | 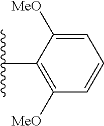 | 0.25 ± 0.22 |
| 15 | 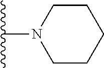 | 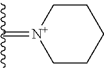 | 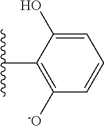 | 2.91 ± 1.86 |
| 16 | 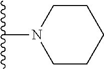 | 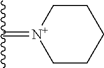 | 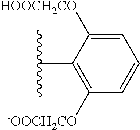 | >100 |

^{a}Mean IC₅₀ and standard deviation of triplicate determination assessed in vitro at 24 h post-treatment using MTT assay.

In vitro antiproliferative assay of compounds 1-12 in HL60. The in vitro antiproliferative activity of compounds 1-12 against a promyelocytic leukemia cell-line, HL60 was determined using a 24 h endpoint MTT assay. Results were expressed as IC₅₀—the concentration of compound (in μM), that inhibits proliferation rate by 50% as compared to control untreated cells. From the assay, compounds 1-3,5,8-14 demonstrated their anti-tumour activity with IC₅₀ values in the sub-micromolar range. Compound 10, which has a thienyl group, and the para-iodo aryl substituted 11 showed the highest activity among the analogues (IC₅₀ of 0.10 and 0.09 μM respectively). In contrast, compounds 4, 6, 7, 15 and 16 displayed moderate to poor activity from single-digit micro-molar IC₅₀ values to undeterminable IC₅₀ up to 100 μM.

The influence of the cyclic amine substituents on the anti-proliferative activity of the compounds was evident from studying compounds 1-6. Regardless of the size of the ring, the derivatives containing hydrophobic cyclic amines from pyrrolidine (1), piperidine (2) to Boc-piperazine (5) exhibited moderate anti-proliferative activity with IC₅₀ values of 0.62-0.76 μM. On the other hand, the derivatives with cyclic amines that contain exposed oxygen or NH isosteres as in the case of compounds 4 and 6 had 10- to 50-fold higher IC₅₀ of 8.27 or 36.7 μM respectively. The unsymmetrical rosamine 3, which had a combination of piperidine and morpholine substituents interestingly had the lowest IC₅₀ value among compounds 1-6, alluding to the possible importance of an ampiphilic structure with contrasting hydrophobic and hydrophilic halves.

For the effect of meso-substitution on anti-proliferative activity of rosamines, compounds 7-16 were studied. Similar to compounds 4 and 6 above, the derivatives with hydrophilic substituents such as the phenolic 15 and the carboxylic 16 had higher IC₅₀ values than the unsubstituted meso-aryl 9. Having an aryl substituent at the meso position, whether directly (9-14) or through an alkyl spacer (8), was important for anti-proliferative activity and was convincingly demonstrated in the lower activity observed in compound 7 which had only a simple methyl substituent at the meso position. Among the aryl substituted compounds, the thiofuran (10) and the para-iodo aryl (11) structures had the lowest IC₅₀ values compared to a simple phenyl-substituted compound 9, while 4-methoxy aryl (12), mono-2-methoxy (13) and di-2-methoxy (14) aryl substitutions did not confer additional activity.

In vitro anti-proliferative activity of compounds 10 & 11 in a panel of cell-lines. The in vitro anti-proliferative activity of the most active compounds 10 and 11 were assessed against a panel of cell-lines derived from human solid tumors including colon cancer, breast cancer, oral squamous cell carcinoma and nasopharyngeal carcinoma (Table 2). A 48 h assay endpoint which is more typical of cytotoxicity studies was used. The anti-proliferative activity of Rh123 was also simultaneously determined for comparison. Both 10 and 11 exhibited at least 10-fold lower IC₅₀ values compared to Rh123. In addition, the thiofuran-substituted compound (10) consistently showed between 1.5-fold to 4-fold lower IC₅₀ values across all 4 types of solid tumors compared to compound 11.

TABLE 2
Cytotoxic effects of rhodamine analogues on carcinoma and immortalized
normal human epithelial cell types.

| | | IC₅₀ (μM) ± S.D.^{a} | | |
|---|---|---|---|---|
| Cell line | Tissue Origin | 10 | 11 | Rh123 |
| HCT116 | Colon | 0.15 ± 0.06 | 0.39 ± 0.11 | 7.92 ± 0.95 |
| MCF-7 | Breast | 0.27 ± 0.16 | 0.39 ± 0.22 | 5.61 ± 0.61 |
| HSC2 | Oral | 0.12 ± 0.09 | 0.25 ± 0.12 | 4.48 ± 2.23 |
| OKF6^{b} | Oral | 0.25 ± 0.10 | 0.41 ± 0.07 | 9.84 ± 3.46 |
| HK1 | Nasopharyngeal | 0.09 ± 0.01 | 0.42 ± 0.06 | 5.86 ± 0.15 |
| NP69^{b} | Nasopharyngeal | 0.33 ± 0.20 | 0.51 ± 0.16 | 6.28 ± 0.21 |

^{a}Mean IC₅₀ and standard deviation of triplicate determination assessed in vitro at 48 h post-treatment using MTT assay.
^{b}Immortalized normal human epithelial cells

To investigate whether rosamines 10 and 11 have greater anti-proliferative effects on cancer cells compared to normal cells, two immortalized epithelial cell-lines from oral (OKF6) and nasopharyngeal (NP69) origin were also included in the study. Gratifyingly, both 10 and 11 were more cytotoxic towards the cancer cell-lines than the immortalized normal cell-lines, as demonstrated in the 1.25-fold to 3-fold higher IC₅₀ values in the normal compared to the cancer cell-lines.

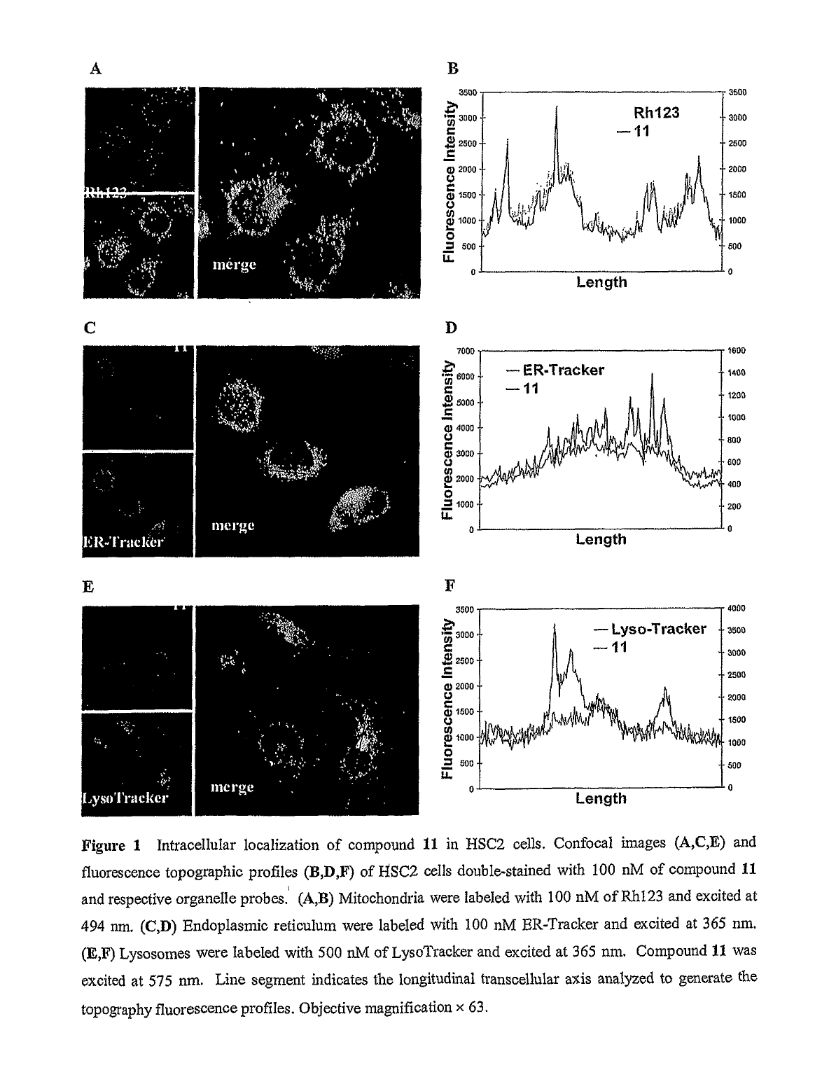

Cellular localization studies. Even though both compound 10 and 11 have equally potent anti-proliferative activity with similar IC₅₀ values in the low sub-micromolar range, the fluorescence quantum yield of 10 is low, at a value that is approximately 3-fold lower compared to 11 (0.28+0.01 vs 0.10+0.01 in ethanol, unpublished data). Therefore, only the intracellular localization of compound 11 in HSC2 cells was examined via confocal microscopy using dual staining techniques (FIG. 1).

Co-staining images and topographic profiles of cells containing compound 11 and a mitochondria-specific dye Rho123 revealed an almost identical overlap, suggesting that compound 11 localised particularly well in mitochondria (FIGS. 1A and B). In comparison, compound 11 displayed only partial co-localisation with ER and lysosomes, according to the confocal images and topographic profiles of compound 11 with ER-Tracker (FIGS. 1C and D) and with LysoTracker (FIGS. 1E and F) respectively. Staining of the cytoplasmic or nuclear membrane by compound 11 was not detected, indicating that compound 11 does not react non-specifically with biological membranes. Furthermore, the nucleus remained free of compound 11 (dark nuclear area) indicating that this class of compounds would not be expected to directly damage DNA.

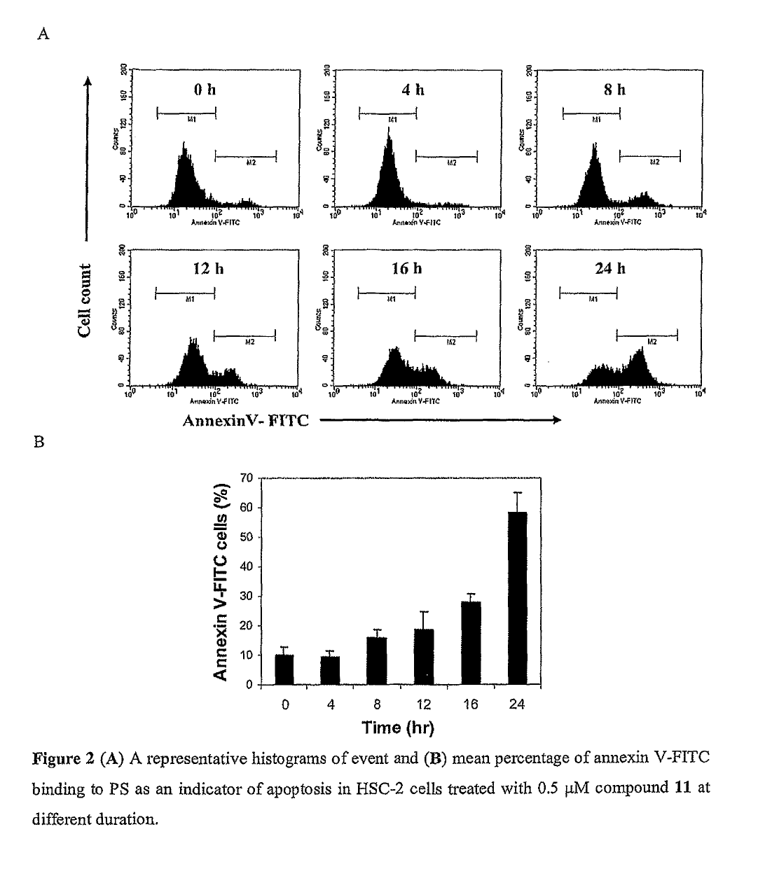

Compound 11 induces apoptosis. The induction of apoptosis was quantified in flow cytometry experiments measuring the externalization of membrane phosphatidylserine through annexin V-FITC staining, which is an event considered characteristic of cells undergoing apoptosis (FIG. 2). Flow cytometric analysis of HSC2 cells treated with compound 11 at IC₅₀ value (0.5 μM) showed the onset of apoptosis at 8 h of compound incubation with 16% of the cells staining positive for annexin V compared to less than 10% at 0 h or 4 h time-points. The proportion of cells undergoing apoptosis continued to increase rapidly to 58% within 24 h.

Compound 11 does not induce cell cycle arrest. The cell cycle profile of HSC2 when treated with an IC₅₀ concentration (0.25 μM) of compound 11 was examined in a time course experiment. From 4 h to 48 h, the cell cycle profile remained unchanged, indicating cell death caused by compound 11 did not occur as a result of cell cycle arrests.

### Conclusions

We have demonstrated the anti-proliferative activity of a new class of rosamine derivatives against a panel of cell-lines from leukemia and solid tumors. Structure activity relationship study indicated the importance of having hydrophobic substituents at the peripheral cyclic amines as well as at the meso-aryl groups. Structures with aryl substituents at the meso position, either directly attached or via a —CH₂— spacer conferred extra activity. The most active compounds 10 and 11 were at least 10-fold more potent than rhodamine123, a structurally similar compound whose anti-cancer properties have been extensively investigated (Modica-Napolitano et al. 1987). Furthermore, our study also showed that compounds 10 and 11 showed greater cytotoxicity towards oral and nasopharyngeal cancer cells compared to immortalized normal cells of the same organ type.

Fluorescence microscopy studies showed that compound 11 localizes exclusively within the mitochondria. This, together with data from cell cycle analysis and onset of apoptosis studies, suggests that compound 11 induced cell death through mitochondria-dependent apoptosis rather than through damage to nucleic materials. The intracellular localization data here also agrees with literature reports where higher mitochondrial transmembrane potential have been noted in cancer cells compared to normal epithelial cells, to result in accumulation of lipophilic cations, such as the rosamine derivatives studied here, in mitochondria (Modica-Napolitano et al. 2001). Overall, our results suggest that these compounds may offer a unique potential for the design of mitochondrial targeting agents that either directly kill or deliver cytotoxic drugs to selectively kill cancer cells.

### Example 4

### Coupling of a Compound of Formula I to Avidin

The water-soluble compound 16 (see Table 1) (6.1 mg, 0.01 mmol) and N-hydroxysuccinimide (1.2 mg, 0.01 mmol) were dissolved in 0.3 mL N,N-dimethylformamide, then N,N′-diisopropylcarbodiimide (1.5 μL, 0.01 mmol) was added. The mixture was stirred at room temperature in the dark for overnight. The solution thus obtained (15 μL, 5 eq.) was added to a solution of avidin (6.6 mg, 1 eq.) in 1 mL sodium bicarbonate buffer (0.1 M, pH 8.3). The mixture was stirred at room temperature in the dark for 1 h. The unreacted dye was removed by PD-10 (Sephadex G-25) column to afford the labelled avidin. The dye:protein ratio was calculated to be 0.8 by UV-Vis when three equivalents of the dye was used; this corresponds to 27% labelling efficiency. When 5 eq. of dye was used then a dye protein ratio of 1.4 (28% labelling efficiency) was observed.

The UV-Vis absorption and fluorescence emission maxima of the 16-avidin conjugate were observed to be within a few nm of compound 16 alone in phosphate buffer. The quantum yield of the protein conjugate was 0.06 in phosphate buffer compared with 0.13 for compound 16 alone.

### REFERENCES

McBride H. M., Neuspiel M., Wasiak S. Mitochondria: More than just a powerhouse. Curr Biol. 2006; 16(14):R551-560.
Johnson L. V., Walsh M. L., Bockus B. J., Chen L. B. Monitoring of relative mitochondria potential in living cells by fluorescence microscopy. J. Cell Biol. 1981; 88:526-535.
Nadakavukaren K. K., Nadakavukaren J. J., Chen L. B. Increased rhodamine 123 uptake by carcinoma cells. Cancer Res. 1985; 45:6093-6099.
Szewczyk A., Wojtczak L. Mitochondria as a pharmacological target. Pharmacol. Rev. 2002; 54:101-127.
Lampidis T. J., Hasin Y., Weiss M. J. Chen L. B. Selective killing of carcinoma cells “in vitro” by lipophilic-cationic compounds: a cellular basis. Biomed. Pharmacother. 1985; 39(5):220-226.
Modica-Napolitano J. S., Aprille J. R. Basis for selective cytotoxicity of rhodamine 123. Cancer Res. 1987; 47:4361-4365.
Lampidis T. J., Bernal S. D., Summerhayes I. C., Chen L. B. Selective toxicity of rhodamine 123 in carcinoma cell in vitro. Cancer Res. 1983; 43(2):716-720.
Modica-Napolitano J. S., Aprille J. R. Delocalized lipophilic cations selectively target the mitochondria of carcinoma cells. Adv. Drug Deliv. Rev. 2001; 49:63-70.
Propper D. J., Braybrooke J. P., Taylor D. J., Lodi R., Styles P., Cramer J. A., Collins W. C., Levitt N. C., Talbot D. C., Ganesan T. S., Harris A. L. Phase I trial of the selective mitochondrial toxin MKT077 in chemo-resistant solid tumours. Ann. Oncol. 1999; 10(8):923-927.
Britten C. D., Rowinsky E. K., Baker S. D., Weiss G. R., Smith L., Stephenson J., Rothenberg M., Smetzer L., Cramer J., Collins W., Von Hoff D. D., Eckhardt S. G. A phase I and pharmacokinetic study of the mitochondrial-specific rhodacyanine dye analog MKT077. Clin. Cancer Res. 2000; 6(1):42-49.
Kawakami M., Koya K., Ukai T., Tatsuta N., Ikegawa A., Ogawa K., Shishido T., Chen L. B. Structure-activity of novel rhodacyanine dyes as antitumor agents. J. Med. Chem. 1998; 41(1):130-42.
Weiss M. J., Wong J. R. Ha C. S., Bleday R. Salem R. R., Steele G. D. Jr., Chen L. B. Dequalinium, a topical antimicrobial agent, displays anticarcinoma activity based on selective mitochondrial accumulation. Proc. Natl. Acad. Sci. USA. 1987; 84(15):5444-5448.
Sun X., Wong J. R., Song K., Hu J., Garlid K. D., Chen L. B. AA1, a newly synthesized monovalent lipophilic cation, expresses potent in vivo antitumor activity. Cancer Res. 1994; 54(6):1465-1471.
Sun X., Wong J. R., Song K., Chen L. B. Anticarcinoma activity of a novel drug, 3-ethyl-3′-methyl-thiatelluracarbocyanine iodide (Te), a tellurium-containing cyanine targeted at mitochondria. 1996; 2(8):1335-1340.
Fantin V. R., Berardi M. J., Scorrano L., Korsmeyer S. J., Leder P. A novel mitochondriotoxic small molecular that selectively inhibits tumor cell growth. 2002; 2:29-42.

1. A compound of formula I: 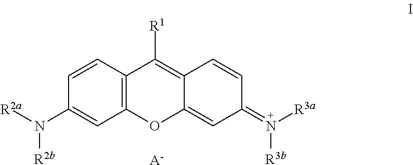
or any pharmaceutically acceptable salt or solvate thereof, wherein:
R¹ represents aryl, Het¹ or C₁₋₆ alkyl, which latter group is optionally substituted by aryl or Het²;
R^{2a} and R^{2b} together form C₃₋₆ n-alkylene, which alkylene group is optionally substituted by one or more substituents selected from halo, C₁₋₄ alkyl, C(O)OH and C(O)O—C₁₋₄ alkyl, and which alkylene group is optionally interrupted by X¹;
R^{3a} and R^{3b} together form C₃₋₆ n-alkylene, which alkylene group is optionally substituted by one or more substituents selected from halo, C₁₋₄ alkyl, C(O)OH and C(O)O—C₁₋₄ alkyl, and which alkylene group is optionally interrupted by X²;
X¹ and X² independently represent O, S, or NR⁴;
R⁴ represents, independently at each occurrence, H, C(O)OR⁵, C(O)R^{6a}, C(O)N(R^{6b})R^{6c} or C₁₋₆ alkyl, which latter group is optionally substituted by one or more substituents selected from halo, aryl and Het³ or is substituted by a single C(O)OR^{4a} group;
R^{4a} represents H or C₁₋₄ alkyl;
R⁵ represents aryl, Het⁴ or C₁₋₆ alkyl optionally substituted by one or more substituents selected from halo, aryl and Het⁵;
R^{6a} to R^{6d} independently represent H or R⁵;
each aryl independently represents a C₆₋₁₀ carbocyclic aromatic group, which group may comprise either one or two rings and may be substituted by one or more substituents selected from halo, CN, C₁₋₆ alkyl (which latter group is optionally substituted by one or more substituents selected from halo, OR⁷, phenyl, naphthyl and Het⁶) and OR^{B};
R⁷ and R⁸ independently represent H, C₁₋₄ alkyl (optionally substituted by one or more halo groups or by a single phenyl or C(O)OR^{8a} substituent), Het⁷, phenyl or naphthyl;
R^{8a} represents H or C₁₋₄ alkyl;
Het¹ to Het⁷ independently represent 5- to 10-membered aromatic, fully saturated or partially unsaturated heterocyclic groups containing one or more heteroatoms selected from oxygen, nitrogen and/or sulfur, which heterocyclic groups may comprise one or two rings and may be substituted by one or more substituents selected from halo, CN, C₁₋₆ alkyl (which latter group is optionally substituted by one or more substituents selected from halo, OR⁹ and phenyl) and OR¹⁰;
R⁹ and R¹⁰ independently represent H, C₁₋₄ alkyl or phenyl;
unless otherwise specified, alkyl groups are optionally substituted by one or more halo atoms; and
A⁻ represents a pharmaceutically acceptable anion.

2. A compound as claimed in claim 1, wherein A⁻ is a chloride ion.

3. A compound as claimed in claim 1 or claim 2, wherein:
R¹ represents methyl, benzyl, phenyl (which latter group is optionally substituted by one or two substituents selected from C₁₋₂ alkyl, halo and C₁₋₂ alkoxy) or thienyl;
R^{2a} and R^{2b} together represent —(CH₂)₄—, —(CH₂)₅— or —(CH₂)₂—X²—(CH₂)₂—;
R^{3a} and R^{3b} together represent —(CH₂)₄—, —(CH₂)₅— or —(CH₂)₂—X²—(CH₂)₂—;
X¹ and X² independently represent O or NR⁴;
R⁴ represents, independently at each occurrence, H or C(O)OR⁵; and
R⁵ represents C₁₋₄ alkyl.

4. A compound of formula I, as defined in claim 1 or claim 2, for use in medicine.

5. A compound of formula I, as defined in claim 1 or claim 2, for use as a dye or chromophore.

6. A pharmaceutical composition comprising a compound of formula I, as defined in claim 1 or claim 2, or any pharmaceutically acceptable salt or solvate thereof, and a pharmaceutically acceptable, carrier, adjuvant or vehicle.

7. A method of treating cancer in a patient in need of such treatment, the method comprising administering to the patient a therapeutically effective amount of a compound of formula I, as defined in claim 1 or claim 2, or any pharmaceutically acceptable salt or solvate thereof.

8. A compound of formula I, as defined in claim 1 or claim 2, or any pharmaceutically acceptable salt or solvate thereof, for use in the treatment of cancer.

9. The use of a compound of formula I, as defined in claim 1 or claim 2, or any pharmaceutically acceptable salt or solvate thereof, for the preparation of a medicament for the treatment of cancer.

10. The method according to claim 7, wherein the cancer is leukemia or a solid tumour cancer.

11. The method, compound for use or the use according to claim 10, wherein the solid tumour cancer is selected from the group consisting of non-small cell lung cancer, small cell lung cancer, breast cancer, nasopharyngeal cancer, oral cancer, cancer of the pancreas, ovarian cancer, colorectal cancer, prostate cancer and gastric cancer, liver cancer, bladder cancer, cancer of the kidney, cervical cancer and cancer of the oesophagus.

12. A combination product comprising a compound of formula I, as defined in claim 1 or claim 2, or any pharmaceutically acceptable salt or solvate thereof, and a known anti-cancer agent.

13. A method of preparing a compound of formula II, 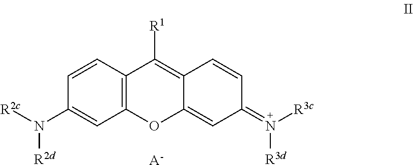
wherein:
R¹ is defined in claim 1;
R^{2c}, R^{2d}, R^{3c} and R^{3d} independently represent C₁₋₆ alkyl (optionally substituted by one or more substituents selected from halo, OR^{a}, N(R^{b})R^{c}, aryl and Het¹,
or R^{2c} and R^{2d} together take the same definition as R^{2a} and R^{2b}, as defined in claim 1 and/or R^{3c} and R^{3d} together take the same definition as R^{3a} and R^{3b}, as defined in claim 1,
R^{a} to R^{c} independently represent H, C₁₋₆ alkyl (optionally substituted by one or more halo groups or by one substituent selected from OH, aryl and Het²), aryl and Het³,
aryl, Het¹ to Het³ and A⁻ are as defined in claim 1,
which process comprises:
(a) reacting a compound of formula III 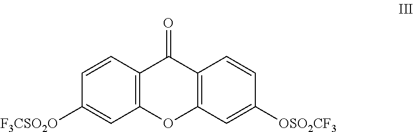
with at least one equivalent each of compounds of formulae IVa and IVb
R^{2c}(R^{2d})N—H IVa
R^{3c}(R^{3d})N—H IVa
wherein R^{2c}, R^{2d}R^{3c} and R^{3d} are as defined above;
(b) reacting the resulting intermediate of formula V 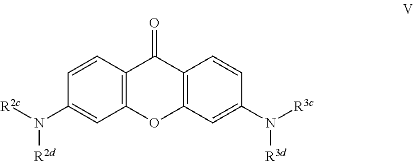
with a compound of formula VIa or VIb
R¹—Mg-Hal VIa
R¹—Li VIb
wherein Hal represents a halogen and R¹ is as defined in claim 1; and then
(c) reacting the resulting intermediate of formula VII 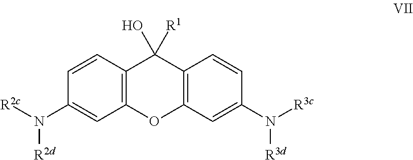
with acid H⁺A⁻, wherein A⁻ is as defined in claim 1.

14. A process for the production of a compound of formula V, as defined in claim 13, said process comprising reacting a compound of formula III, as defined in claim 13, with at least one equivalent each of compounds of formulae IVa and IVb, as defined in claim 13.

15. A process for the preparation of a compound of formula IIIa, 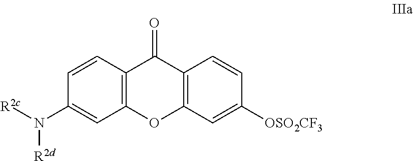
wherein R^{2c} and R^{2d} are as defined in claim 13,
said process comprising reacting a compound of formula III, as defined in claim 13, with at least one equivalent of a compound of formula IVa, as defined in claim 13.

16. A process for the preparation of a compound of formula IIIb, 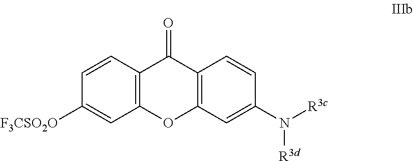
wherein R^{3c} and R^{3d} are as defined in claim 13,
said process comprising reacting a compound of formula III, as defined in claim 13, with at least one equivalent of a compound of formula IVb, as defined in claim 13.

17. A compound of formula IIIa, as defined in claim 15.

18. A compound of formula IIIb, as defined in claim 16.

19. The compound for use according to claim 8 wherein the cancer is leukemia or a solid tumour cancer.

20. The use according to claim 1, wherein the cancer is leukemia or a solid tumour cancer.
